(19) United States
(12) Patent Application Publication
(10) Pub. No.: US 2016/0271086 A1
(43) Pub. Date: Sep. 22, 2016

(54) METHODS AND COMPOSITIONS FOR INDUCING REGULATORY T-CELL GENERATION

(71) Applicant: SLOAN-KETTERING INSTITUTE FOR CANCER RESEARCH, New York, NY (US)

(72) Inventors: Alexander Rudensky, New York, NY (US); Nicholas Arpaia, New York, NY (US)

(21) Appl. No.: 15/033,145

(22) PCT Filed: Oct. 31, 2014
(86) PCT No.: PCT/US14/63354

§ 371 Date: Apr. 29, 2016

Related U.S. Application Data
(60) Provisional application No. 61/898,242, filed on Oct. 31, 2013.

(51) Int. Cl. A61K 31/19; G01N 33/50

(52) U.S. Cl. CPC A61K 31/19; G01N 33/505; G01N 33/5023

(57) ABSTRACT

The present invention provides, among other things, methods and compositions for modulating inflammation. The present invention is based, in part, on the surprising discovery that peripheral regulatory T cells (pTreg), can be induced to differentiate after exposure to bacterial metabolites. In some embodiments, provided methods and compositions are used to treat diseases resulting from inflammation.

### CROSS REFERENCE TO RELATED APPLICATIONS

This application claims the benefit of U.S. Provisional Application Ser. No. 61/898,242, filed Oct. 31, 2013, the disclosure of which is hereby incorporated by reference.

### BACKGROUND

Inflammation is a major part of the biological response to damaged cells, irritants and pathogens. There is an ongoing need to better understand this complex process in the hope of discovering new treatments. New developments in inflammation research will lead to better treatments for disorders resulting from this process. There is a great deal of effort aimed at developing compositions and methods for treating diseases related to inflammation.

### SUMMARY

The present invention provides, among other things, methods and compositions for the treatment of inflammation. The present invention is based, in part, on the surprising discovery that bacterial metabolites, particularly short chain fatty acids, can promote generation of T regulatory (pTreg) cells.

In some embodiments, the present invention provides methods of inducing differentiation of regulatory T cells (Treg), the method comprising steps of: administering to cells undergoing inflammation a composition comprising a histone deacetylase (HDAC) inhibitory agent. In some embodiments, the step of administering comprises administering to dendritic cells and naïve CD4⁺ T cells a composition comprising a histone deacetylase (HDAC) inhibitory agent. In some embodiments, the step of administering comprises administering a composition comprising a histone deacetylase (HDAC) inhibitory agent that acts to promote Treg differentiation in a Foxp3-dependent manner.

In some embodiments, the step of administering comprises administering a composition selected from the group comprising bacterial metabolites, short chain fatty acids, histone deacetylase (HDAC) inhibitors, and analogs thereof. In some embodiments, the step of administering comprises administering a composition comprising short chain fatty acids. In some embodiments, the step of administering comprises administering short chain fatty acids selected from the group comprising butyrate, propionate, succinate, formate, valproate, phenylbutyrate, L-lactate, 2-ethylbutyrate, isovalerate, isobutyric acid, valeric acid, acetate and analogs thereof.

In some embodiments, the step of administering comprises administering to a site or individual suffering from or susceptible to inflammation and furthermore comprises administering the composition by a route according to a schedule so that inflammation is reduced. In some embodiments, the step of administering comprises administering to a subject suffering from or susceptible to a Treg-associated Disease, Disorder or Condition. In some embodiments, the Treg-associated Disease, Disorder or Condition is selected from the group comprising colitis, asthma, chronic peptic ulcer, tuberculosis, rheumatoid arthritis, chronic periodontitis, Crohn's disease, chronic sinusitis, pelvic inflammatory disease, hepatitis, inflammatory bowel disease, sarcoidosis, vasculitis, celiac disease, autoimmune disease, reperfusion injury, transplant rejection, diabetes and infection.

In some embodiments, the step of administering comprises administering by a route selected from intradermal, intramuscular, intraoperative, intrathecal, intravenous, nasal, ocular, oral, parental, rectal, subcutaneous, topical, and transdermal.

In some embodiments, the invention is a method comprising steps of: administering to a subject in need thereof a pharmaceutical composition comprising an activating agent that is or increases the level of a short chain fatty acid to induce Treg differentiation in a subject.

In some embodiments, the invention is a method comprising the steps of: administering to a subject in need thereof a pharmaceutical composition comprising an activating agent that is or increases the level of a bacterial metabolite selected from the group consisting of short chain fatty acid. In some embodiments, the pharmaceutical composition delivers an amount of the active agent that is effective, when the composition is administered in accordance with a therapeutic regimen, to increase level or activity of pTreg cells. In some embodiments, the increase level or activity of pTreg cells occurs at local sites of pTreg cells. In some embodiments, the increase level or activity of pTreg cells occurs in pTregs systemically throughout the organism. In some embodiments, the step of administering comprises administering compositions to treat an animal. In some embodiments, the step of administering comprises administering compositions to treat a mammal.

In some embodiments, the invention is a method of identifying or characterizing a pTreg stimulatory agent, the method comprising steps of: administering the candidate pTreg stimulatory agent to a T cell population; comparing the pTreg level and/or activity to a reference set of positive control conditions and negative control conditions; and determining whether the pTreg level and/or activity is at least comparable to positive control conditions and/or higher than that under (other) negative control conditions.

In some embodiments, the invention is an agent characterized in that when administered to T cells will induce differentiation of Treg cells. In some embodiments, the agent acts to promote Treg differentiation in a Foxp3-dependent manner. In some embodiments, the agent is selected from a group comprising bacterial metabolites, short chain fatty acids, histone deacetylase (HDAC) inhibitors and analogs (e.g. TSA) thereof. In some embodiments, the agent is a short chain fatty acid selected from the group comprising butyrate, propionate, succinate, formate, valproate, phenylbutyrate, L-lactate, 2-ethylbutyrate, isovalerate, isobutyric acid, valeric acid, acetate, and pharmaceutically acceptable salts and analogs thereof.

As used in this application, the terms “about” and “approximately” are used as equivalents. Any numerals used in this application with or without about/approximately are meant to cover any normal fluctuations appreciated by one of ordinary skill in the relevant art.

Other features, objects, and advantages of the present invention are apparent in the detailed description that follows. It should be understood, however, that the detailed description, while indicating embodiments of the present invention. is given by way of illustration only, not limitation. Various changes and modifications within the scope of the invention will become apparent to those skilled in the art from the detailed description.

### DEFINITIONS

In order for the present invention to be more readily understood, certain terms are first defined below. Additional definitions for the following terms and other terms are set forth throughout the specification.

Activates: As used herein, the term “activates” refers to increasing the level or activity of a target.

Activating agent: As used herein, the term “activating agent” refers to an agent whose presence or level correlates with elevated level or activity of a target, as compared with that observed absent the agent (or with the agent at a different level). In some embodiments, an activating agent is one whose presence or level correlates with a target level or activity that is comparable to or greater than a particular reference level or activity (e.g., that observed under appropriate reference conditions, such as presence of a known activating agent, e.g., a positive control).

Affinity: As is known in the art, “affinity” is a measure of the tightness with a particular ligand binds to its partner. Affinities can be measured in different ways. In some embodiments, affinity is measured by a quantitative assay. In some such embodiments, binding partner concentration may be fixed to be in excess of ligand concentration so as to mimic physiological conditions. Alternatively or additionally, in some embodiments, binding partner concentration and/or ligand concentration may be varied. In some such embodiments, affinity may be compared to a reference under comparable conditions (e.g., concentrations).

Agent: The term “agent” as used herein may refer to a compound or entity of any chemical class including, for example, polypeptides, nucleic acids, saccharides, lipids, small molecules, metals, or combinations thereof. As will be clear from context, in some embodiments, an agent can be or comprise a cell or organism, or a fraction, extract, or component thereof. In some embodiments, an agent is agent is or comprises a natural product in that it is found in and/or is obtained from nature. In some embodiments, an agent is or comprises one or more entities that is man-made in that it is designed, engineered, and/or produced through action of the hand of man and/or is not found in nature. In some embodiments, an agent may be utilized in isolated or pure form; in some embodiments, an agent may be utilized in crude form. In some embodiments, potential agents are provided as collections or libraries, for example that may be screened to identify or characterize active agents within them. Some particular embodiments of agents that may be utilized in accordance with the present invention include small molecules, antibodies, antibody fragments, aptamers, siRNAs, shRNAs, DNA/RNA hybrids, antisense oligonucleotides, ribozymes, peptides, peptide mimetics, small molecules, etc. In some embodiments, an agent is or comprises a polymer. In some embodiments, an agent is not a polymer and/or is substantially free of any polymer. In some embodiments, an agent contains at least one polymeric moiety. In some embodiments, an agent lacks or is substantially free of any polymeric moiety.

Analog: As used herein, the term “analog” refers to a substance that shares one or more particular structural features, elements, components, or moieties with a reference substance. Typically, an “analog” shows significant structural similarity with the reference substance, for example sharing a core or consensus structure, but also differs in certain discrete ways. In some embodiments, an analog a substance that can be generated from the reference substance by chemical manipulation of the reference substance. In some embodiments, an analog is a substance that can be generated through performance of a synthetic process substantially similar to (e.g., sharing a plurality of steps with) one that generates the reference substance. In some embodiments, an analog is or can be generated through performance of a synthetic process different from that used to generate the reference substance.

Animal: As used herein, the term “animal” refers to any member of the animal kingdom. In some embodiments, “animal” refers to humans, at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, insects, and/or worms. In some embodiments, an animal may be a transgenic animal, genetically-engineered animal, and/or a clone.

Antagonist: As used herein, the term “antagonist” refers to an agent that i) inhibits, decreases or reduces level and/or activity of another entity and/or ii) inhibits, decreases, delays or reduces one or more effects of such other entity; and/or ii) inhibits, decreases, reduces, or delays one or more biological events. Antagonists may be or include agents of any chemical class including, for example, small molecules, polypeptides, nucleic acids, carbohydrates, lipids, metals, and/or any other entity that shows the relevant inhibitory activity. In some embodiments, an antagonist may be direct (in which case it exerts its influence directly upon its target); in some embodiments, an antagonist may be indirect (in which case it exerts its influence by other than binding to its target; e.g., by interacting with a regulator of the target, for example so that level or activity of the target is altered). In some embodiments, action of an antagonist may be reversible; in some embodiments it may be irreversible. In some embodiments, an antagonist may form a covalent bond with its target; in many such embodiments, the antagonist acts as an irreversible inhibitor of that target. In some embodiments, an antagonist interacts with an active site on its target (e.g., a site of interaction with a partner entity or substrate). In some embodiments, an antagonist competes with another entity (e.g., a partner binding agent or a substrate) for interaction with a target.

Antigen: As used herein, the term “antigen” refers to a molecule or entity to which an antibody binds. In some embodiments, an antigen is or comprises a polypeptide or portion thereof. In some embodiments, an antigen is an agent that elicits an immune response; and/or (ii) an agent that is bound by a T cell receptor (e.g., when presented by an MHC molecule) or to an antibody (e.g., produced by a B cell) when exposed or administered to an organism. In some embodiments, an antigen elicits a humoral response (e.g., including production of antigen-specific antibodies) in an organism; alternatively or additionally, in some embodiments, an antigen elicits a cellular response (e.g., involving T-cells whose receptors specifically interact with the antigen) in an organism. It will be appreciated by those skilled in the art that a particular antigen may elicit an immune response in one or several members of a target organism (e.g., mice, rabbits, primates, humans), but not in all members of the target organism species. In some embodiments, an antigen elicits an immune response in at least about 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% of the members of a target organism species. In some embodiments, an antigen binds to an antibody and/or T cell receptor, and may or may not induce a particular physiological response in an organism. In some embodiments, for example, an antigen may bind to an antibody and/or to a T cell receptor in vitro, whether or not such an interaction occurs in vivo. In general, an antigen may be or include any chemical entity such as, for example, a small molecule, a nucleic acid, a polypeptide, a carbohydrate, a lipid, a polymer [in some embodiments other than a biologic polymer (e.g., other than a nucleic acid or amino acid polymer)] etc. In some embodiments, an antigen is or comprises a polypeptide. In some embodiments, an antigen is or comprises a glycan. Those of ordinary skill in the art will appreciate that, in general, an antigen may be provided in isolated or pure form, or alternatively may be provided in crude form (e.g., together with other materials, for example in an extract such as a cellular extract or other relatively crude preparation of an antigen-containing source). In some embodiments, antigens utilized in accordance with the present invention are provided in a crude form. In some embodiments, an antigen is or comprises a recombinant antigen.

Approximately or about: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Binding: As used herein, the term “binding” refers to a non-covalent association between or among two or more entities. “Direct” binding involves physical contact between entities or moieties; indirect binding involves physical interaction by way of physical contact with one or more intermediate entities. Binding between two or more entities can be assessed in any of a variety of contexts—including where interacting entities or moieties are studied in isolation or in the context of more complex systems (e.g., while covalently or otherwise associated with a carrier entity and/or in a biological system or cell).

Biologically active: As used herein, the phrase “biologically active” refers to a characteristic of any agent that has activity in a biological system, and particularly in an organism. For instance, an agent that, when administered to an organism, has a biological effect on that organism, is considered to be biologically active. In particular embodiments, where a peptide is biologically active, a portion of that peptide that shares at least one biological activity of the peptide is typically referred to as a “biologically active” portion. In certain embodiments, a peptide has no intrinsic biological activity but that inhibits the effects of one or more naturally-occurring angiotensin compounds is considered to be biologically active.

Carrier or diluent: As used herein, the terms “carrier” and “diluent” refers to a pharmaceutically acceptable (e.g., safe and non-toxic for administration to a human) carrier or diluting substance useful for the preparation of a pharmaceutical formulation. Exemplary diluents include sterile water, bacteriostatic water for injection (BWFI), a pH buffered solution (e.g., phosphate-buffered saline), sterile saline solution, Ringer's solution or dextrose solution.

Characteristic portion: As used herein, the term a “characteristic portion” of a substance, in the broadest sense, is one that shares some degree of sequence or structural identity with respect to the whole substance. In certain embodiments, a characteristic portion shares at least one functional characteristic with the intact substance. For example, a “characteristic portion” of a protein or polypeptide is one that contains a continuous stretch of amino acids, or a collection of continuous stretches of amino acids, that together are characteristic of a protein or polypeptide. In some embodiments, each such continuous stretch generally contains at least 2, 5, 10, 15, 20, 50, or more amino acids. In general, a characteristic portion of a substance (e.g., of a protein, antibody, etc.) is one that, in addition to the sequence and/or structural identity specified above, shares at least one functional characteristic with the relevant intact substance; epitope-binding specificity is one example. In some embodiments, a characteristic portion may be biologically active.

Combination therapy: As used herein, the term “combination therapy” refers to those situations in which two or more different pharmaceutical agents for the treatment of disease are administered in overlapping regimens so that the subject is simultaneously exposed to at least two agents. In some embodiments, the different agents are administered simultaneously. In some embodiments, the administration of one agent overlaps the administration of at least one other agent. In some embodiments, the different agents are administered sequentially such that the agents have simultaneous biologically activity with in a subject.

Comparable: The term “comparable” is used herein to describe two (or more) sets of conditions, circumstances, individuals, or populations that are sufficiently similar to one another to permit comparison of results obtained or phenomena observed. In some embodiments, comparable sets of conditions, circumstances, individuals, or populations are characterized by a plurality of substantially identical features and one or a small number of varied features. Those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

Dendritic cell: As used herein, the term “dendritic cell” refers to immune cells whose main function is to process antigen material and present it on the surface to other cells of the immune system. Dendritic cells act as messengers between the innate and adaptive immunity and are communicating with other cells through direct contact or at a distance using cytokines Reacting to the presence of foreign antigens, dendritic cells produce cytokines which in turn induce other immune cells, T cells for example, to aid in the immune response.

Diagnostic information: As used herein, the term “diagnostic information” or information for use in diagnosis is any information that is useful in determining whether a patient has a disease or condition and/or in classifying the disease or condition into a phenotypic category or any category having significance with regard to prognosis of the disease or condition, or likely response to treatment (either treatment in general or any particular treatment) of the disease or condition. Similarly, diagnosis refers to providing any type of diagnostic information, including, but not limited to, whether a subject is likely to have a disease or condition (such as cancer), state, staging or characteristic of the disease or condition as manifested in the subject, information related to the nature or classification of a tumor, information related to prognosis and/or information useful in selecting an appropriate treatment. Selection of treatment may include the choice of a particular therapeutic (e.g., chemotherapeutic) agent or other treatment modality such as surgery, radiation, etc., a choice about whether to withhold or deliver therapy, a choice relating to dosing regimen (e.g., frequency or level of one or more doses of a particular therapeutic agent or combination of therapeutic agents), etc.

Dosage form: As used herein, the terms “dosage form” and “unit dosage form” refer to a physically discrete unit of a therapeutic agent for the patient to be treated. Each unit contains a predetermined quantity of active material calculated to produce the desired therapeutic effect. It will be understood, however, that the total dosage of the composition will be decided by the attending physician within the scope of sound medical judgment.

Dosing regimen: A “dosing regimen” (or “therapeutic regimen”), as that term is used herein, is a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which are separated from one another by a time period of the same length; in some embodiments, a dosing regimen comprises a plurality of doses and at least two different time periods separating individual doses. In some embodiments, the therapeutic agent is administered continuously over a predetermined period. In some embodiments, the therapeutic agent is administered once a day (QD) or twice a day (BID).

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end formation); (3) translation of an RNA into a polypeptide or protein; and/or (4) post-translational modification of a polypeptide or protein.

Functional: As used herein, a “functional” biological molecule is a biological molecule in a form in which it exhibits a property and/or activity by which it is characterized. A biological molecule may have two functions (i.e., bifunctional) or many functions (i.e., multifunctional).

Functional equivalent or analog: As used herein, the term “functional equivalent” or “functional analog” denotes, in the context of a functional analog of an amino acid sequence, a molecule that retains a biological activity (either function or structural) that is substantially similar to that of the original sequence. A functional analog or equivalent may be a natural analog or is prepared synthetically. Exemplary functional analogs include amino acid sequences having substitutions, deletions, or additions of one or more amino acids, provided that the biological activity of the protein is conserved. The substituting amino acid desirably has chemico-physical properties which are similar to that of the substituted amino acid. Desirable similar chemico-physical properties include, similarities in charge, bulkiness, hydrophobicity, hydrophilicity, and the like.

Gene: As used herein, the term “gene” has its meaning as understood in the art. In some embodiments, the term “gene” may include gene regulatory sequences (e.g., promoters, enhancers, etc.) and/or intron sequences. In some embodiments, the term refers to nucleic acids that do not encode proteins but rather encode functional RNA molecules such as tRNAs, RNAi-inducing agents, etc. Alternatively or additionally, in many embodiments, the term “gene”, as used in the present application, refers to a portion of a nucleic acid that encodes a protein. Whether the term encompasses other sequences (e.g., non-coding sequences, regulatory sequences, etc.) will be clear from context to those of ordinary skill in the art.

Gene product or expression product: As used herein, the term “gene product” or “expression product” generally refers to an RNA transcribed from the gene (pre- and/or post-processing) or a polypeptide (pre- and/or post-modification) encoded by an RNA transcribed from the gene.

HDAC inhibitory agent: As used herein, the term “HDAC inhibitory agent” refers to a composition that acts as an antagonist of one or more histone deacetylases.

Immunotherapy: as used herein the term “immunotherapy” refers to treatment of a disease, disorder or condition by inducing, enhancing, or suppressing an immune response. In some embodiments, the relevant immune response may be or include an active response; in some embodiments, the relevant immune response may be or include a passive, response. In some embodiments, the relevant immune response may be or include a Th1 response; in some embodiments, the relevant immune response may be or include a Th2 response. Those of ordinary skill in the art will appreciate that different embodiments of the present invention may implicate or involve different types of immune reactions.

Improve, increase, or reduce: As used herein, the terms “improve,” “increase” or “reduce,” or grammatical equivalents, indicate values that are relative to an appropriate baseline or reference level or amount. Those of ordinary skill in the art will be aware of appropriate reference levels or amount for particular values of interest in accordance with the present invention. To give but a few examples, in some embodiments, a reference level or amount is that determined under otherwise comparable conditions (e.g., in the same system or individual) absent administration of a particular agent. In some embodiments a reference level or amount is that determined in an appropriate comparator system, individual, or population (e.g., in a system, individual or population not afflicted with or representative of a particular disease, disorder or condition).

In vitro: As used herein, the term “in vitro” refers to events that occur in an artificial environment, e.g., in a test tube or reaction vessel, in cell culture, etc., rather than within a multi-cellular organism.

In vivo: As used herein, the term “in vivo” refers to events that occur within a multi-cellular organism, such as a human and a non-human animal. In the context of cell-based systems, the term may be used to refer to events that occur within a living cell (as opposed to, for example, in vitro systems).

Inflammation: As used herein, the term “inflammation” refers to the localized protective response of vascular tissues to injury, irritation or infection. In some embodiments, an inflammatory condition is characterized by one or more of the following symptoms: redness, swelling, pain and loss of function.

Isolated: As used herein, the term “isolated” refers to a substance and/or entity that has been (1) separated from at least some of the components with which it was associated when initially produced (whether in nature and/or in an experimental setting), and/or (2) produced, prepared, and/or manufactured by the hand of man. Isolated substances and/or entities may be separated from at least about 10%, about 20%, about 30%, about 40%, about 50%, about 60%, about 70%, about 80%, about 90%, about 95%, about 98%, about 99%, substantially 100%, or 100% of the other components with which they were initially associated. In some embodiments, isolated agents are more than about 80%, about 85%, about 90%, about 91%, about 92%, about 93%, about 94%, about 95%, about 96%, about 97%, about 98%, about 99%, substantially 100%, or 100% pure. As used herein, a substance is “pure” if it is substantially free of other components. As used herein, the term “isolated cell” refers to a cell that is not presently part of a multi-cellular organism.

Patient: As used herein, the term “patient” or “subject” refers to any organism to which a provided composition is or may be administered, e.g., for experimental, diagnostic, prophylactic, cosmetic, and/or therapeutic purposes. Typical patients include animals (e.g., mammals such as mice, rats, rabbits, non-human primates, and/or humans). In some embodiments, a patient is a human. In some embodiments, a patient is suffering from or susceptible to one or more disorders or conditions. In some embodiments, a patient displays one or more symptoms of a disorder or condition. In some embodiments, a patient has been diagnosed with one or more disorders or conditions. In some embodiments, the disorder or condition is or includes inflammation.

Pharmaceutically acceptable: As used herein, the term “pharmaceutically acceptable”, refers to substances that, within the scope of sound medical judgment, are suitable for use in contact with the tissues of human beings and animals without excessive toxicity, irritation, allergic response, or other problem or complication, commensurate with a reasonable benefit/risk ratio.

Pharmaceutical composition: As used herein, the term “pharmaceutical composition” refers to an active agent, formulated together with one or more pharmaceutically acceptable carriers. In some embodiments, active agent is present in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam, enema; sublingually; ocularly; transdermally; or nasally, pulmonary, and to other mucosal surfaces.

Pharmaceutically acceptable: The term “pharmaceutically acceptable” as used herein, refers to substances that, within the scope of sound medical judgment, are suitable for use in contact with the tissues of human beings and animals without excessive toxicity, irritation, allergic response, or other problem or complication, commensurate with a reasonable benefit/risk ratio.

Prevent: As used herein, the term “prevent” or “prevention”, when used in connection with the occurrence of a disease, disorder, and/or condition, refers to reducing the risk of developing the disease, disorder and/or condition. See the definition of “risk.”

Polypeptide: The term “polypeptide” as used herein refers a sequential chain of amino acids linked together via peptide bonds. The term is used to refer to an amino acid chain of any length, but one of ordinary skill in the art will understand that the term is not limited to lengthy chains and can refer to a minimal chain comprising two amino acids linked together via a peptide bond. As is known to those skilled in the art, polypeptides may be processed and/or modified.

Prognostic and predictive information: As used herein, the terms “prognostic and predictive information” are used interchangeably to refer to any information that may be used to indicate any aspect of the course of a disease or condition either in the absence or presence of treatment. Such information may include, but is not limited to, the average life expectancy of a patient, the likelihood that a patient will survive for a given amount of time (e.g., 6 months, 1 year, 5 years, etc.), the likelihood that a patient will be cured of a disease, the likelihood that a patient's disease will respond to a particular therapy (wherein response may be defined in any of a variety of ways). Prognostic and predictive information are included within the broad category of diagnostic information.

Protein: The term “protein” as used herein refers to one or more polypeptides that function as a discrete unit. If a single polypeptide is the discrete functioning unit and does not require permanent or temporary physical association with other polypeptides in order to form the discrete functioning unit, the terms “polypeptide” and “protein” may be used interchangeably. If the discrete functional unit is comprised of more than one polypeptide that physically associate with one another, the term “protein” refers to the multiple polypeptides that are physically coupled and function together as the discrete unit.

Reference: The term “reference” is often used herein to describe a standard or control agent, individual, population, sample, sequence or value against which an agent, individual, population, sample, sequence or value of interest is compared. In some embodiments, a reference agent, individual, population, sample, sequence or value is tested and/or determined substantially simultaneously with the testing or determination of the agent, individual, population, sample, sequence or value of interest. In some embodiments, a reference agent, individual, population, sample, sequence or value is a historical reference, optionally embodied in a tangible medium. Typically, as would be understood by those skilled in the art, a reference agent, individual, population, sample, sequence or value is determined or characterized under conditions comparable to those utilized to determine or characterize the agent, individual, population, sample, sequence or value of interest

Response: As used herein, a “response” to treatment may refer to any alteration in a subject's condition that occurs as a result of or correlates with treatment. Such alteration may include a beneficial alteration, such as stabilization of the condition (e.g., prevention of deterioration that would have taken place in the absence of the treatment), amelioration of symptoms of the condition, and/or improvement in the prospects for cure of the condition, etc. It may refer to a subject's response. Subject response may be measured according to a wide variety of criteria, including clinical criteria and objective criteria. In some embodiments, a response may include an alteration that is not beneficial (e.g., a side effect).

Risk: As will be understood from context, a “risk” of a disease, disorder, and/or condition comprises a likelihood that a particular individual will develop a disease, disorder, and/or condition (e.g., cancer). In some embodiments, risk is expressed as a percentage. In some embodiments, risk is from 0, 1, 2, 3, 4, 5, 6, 7, 8, 9, 10, 20, 30, 40, 50, 60, 70, 80, 90 up to 100%. In some embodiments risk is expressed as a risk relative to a risk associated with a reference sample or group of reference samples. In some embodiments, a reference sample or group of reference samples have a known risk of a disease, disorder, condition and/or event (e.g., cancer). In some embodiments a reference sample or group of reference samples are from individuals comparable to a particular individual. In some embodiments, relative risk is 0, 1, 2, 3, 4, 5, 6, 7, 8, 9, 10, or more.

Sample: As used herein, a sample obtained from a subject may include, but is not limited to, one or more of the following: a cell or cells, a portion of tissue, blood, serum, ascites, urine, saliva, and other body fluids, secretions, or excretions. The term “sample” also includes any material derived by processing such a sample. Derived samples may include nucleotide molecules or polypeptides extracted from the sample or obtained by subjecting the sample to techniques such as amplification or reverse transcription of mRNA, etc.

Specific binding: As used herein, the terms “specific binding” or “specific for” or “specific to” refer to an interaction (typically non-covalent) between a target entity (e.g., a target protein or polypeptide) and a binding agent (e.g., an antibody, such as a provided antibody). As will be understood by those of ordinary skill, an interaction is considered to be “specific” if it is favored in the presence of alternative interactions. In many embodiments, an interaction is typically dependent upon the presence of a particular structural feature of the target molecule such as an antigenic determinant or epitope recognized by the binding molecule. For example, if an antibody is specific for epitope A, the presence of a polypeptide containing epitope A or the presence of free unlabeled A in a reaction containing both free labeled A and the antibody thereto, will reduce the amount of labeled A that binds to the antibody. It is to be understood that specificity need not be absolute. For example, it is well known in the art that numerous antibodies cross-react with other epitopes in addition to those present in the target molecule. Such cross-reactivity may be acceptable depending upon the application for which the antibody is to be used. Specificity may be evaluated in the context of additional factors such as the affinity of the binding molecule for the target molecule versus the affinity of the binding molecule for other targets (e.g., competitors). If a binding molecule exhibits a high affinity for a target molecule that it is desired to detect and low affinity for non-target molecules, the antibody will likely be an acceptable reagent for immunodiagnostic purposes. Once the specificity of a binding molecule is established in one or more contexts, it may be employed in other, preferably similar, contexts without necessarily re-evaluating its specificity.

Subject: As used herein, the term “subject” refers to a human or any non-human animal (e.g., mouse, rat, rabbit, dog, cat, cattle, swine, sheep, horse or primate). A human includes pre- and post-natal forms. In many embodiments, a subject is a human being. A subject can be a patient, which refers to a human presenting to a medical provider for diagnosis or treatment of a disease. The term “subject” is used herein interchangeably with “individual” or “patient.” A subject can be afflicted with or is susceptible to a disease or disorder but may or may not display symptoms of the disease or disorder.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Suffering from: An individual who is “suffering from” a disease, disorder, and/or condition has been diagnosed with or displays one or more symptoms of the disease, disorder, and/or condition.

Susceptible to: An individual who is “susceptible to” a disease, disorder, and/or condition has not been diagnosed with the disease, disorder, and/or condition. In some embodiments, an individual who is susceptible to a disease. disorder, and/or condition may not exhibit symptoms of the disease, disorder, and/or condition. In some embodiments, an individual who is susceptible to a disease, disorder, condition, or event (for example, cancer) may be characterized by one or more of the following: (1) a genetic mutation associated with development of the disease, disorder, and/or condition; (2) a genetic polymorphism associated with development of the disease, disorder, and/or condition; (3) increased and/or decreased expression and/or activity of a protein associated with the disease, disorder, and/or condition; (4) habits and/or lifestyles associated with development of the disease, disorder, condition, and/or event. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Symptoms are reduced: According to the present invention, “symptoms are reduced” when one or more symptoms of a particular disease, disorder or condition is reduced in magnitude (e.g., intensity, severity, etc.) and/or frequency. For purposes of clarity, a delay in the onset of a particular symptom is considered one form of reducing the frequency of that symptom. Many cancer patients with smaller tumors have no symptoms. It is not intended that the present invention be limited only to cases where the symptoms are eliminated. The present invention specifically contemplates treatment such that one or more symptoms is/are reduced (and the condition of the subject is thereby “improved”), albeit not completely eliminated.

T cell: As used herein, the term “T cell” refers to lymphocytes (white blood cells) that function in cell-mediated immunity. The presence of a T cell receptor (TCR) on the cell surface distinguishes them from other lymphocytes. T cells do not present antigens and rely on other lymphocytes (natural killer cells, B cells, macrophages, dendritic cells) to aid in antigen presentation. Types of T cells include: T helper cells (TH cells), Memory T cells (Tcm, Tem, or Temra), Regulatory T cells (Treg), Cytotoxic T cells (CTLs), Natural killer T cells (NKT cells), gamma delta T cells, and Mucosal associated invariant T cells (MALT).

Therapeutic agent: As used herein, the phrase “therapeutic agent” refers to any agent that has a therapeutic effect and/or elicits a desired biological and/or pharmacological effect, when administered to a subject.

Therapeutically effective amount: As used herein, the term “therapeutically effective amount” refers to an amount of a therapeutic agent (e.g., bacterial metabolites, short chain fatty acids, HDAC inhibitors) which confers a therapeutic effect on the treated subject, at a reasonable benefit/risk ratio applicable to any medical treatment. The therapeutic effect may be objective (i.e., measurable by some test or marker) or subjective (i.e., subject gives an indication of or feels an effect). In particular, the “therapeutically effective amount” refers to an amount of a therapeutic protein or composition effective to treat, ameliorate, or prevent a desired disease or condition, or to exhibit a detectable therapeutic or preventative effect, such as by ameliorating symptoms associated with the disease, preventing or delaying the onset of the disease, and/or also lessening the severity or frequency of symptoms of the disease. A therapeutically effective amount is commonly administered in a dosing regimen that may comprise multiple unit doses. For any particular therapeutic protein, a therapeutically effective amount (and/or an appropriate unit dose within an effective dosing regimen) may vary, for example, depending on route of administration, on combination with other pharmaceutical agents. Also, the specific therapeutically effective amount (and/or unit dose) for any particular patient may depend upon a variety of factors including the disorder being treated and the severity of the disorder; the activity of the specific pharmaceutical agent employed; the specific composition employed; the age, body weight, general health, sex and diet of the patient; the time of administration, route of administration, and/or rate of excretion or metabolism of the specific fusion protein employed; the duration of the treatment; and like factors as is well known in the medical arts.

Treating: As used herein, the term “treat,” “treatment,” or “treating” refers to any method used to partially or completely alleviate, ameliorate, relieve, inhibit, prevent, delay onset of, reduce severity of and/or reduce incidence of one or more symptoms or features of a particular disease, disorder, and/or condition. Treatment may be administered to a subject who does not exhibit signs of a disease and/or exhibits only early signs of the disease for the purpose of decreasing the risk of developing pathology associated with the disease. Alternatively or additionally, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

Treg cells: As used herein, the term Treg cell refers to Regulatory T cells (Treg) formerly known as Suppressor T cells. Treg cells maintain immunological tolerance. During an immune response, Tregs stop T cell-mediated immunity and suppress auto-reactive T cells that have escaped negative selection within the thymus. Adaptive Treg cells (called Th3 or Tr 1 cells) are thought to be generated during an immune response. Naturally occurring Treg cells (CD4+CD25+FoxP3+ Treg cells) are generated in the thymus and have been linked to interactions between developing T cells with both myeloid (CD11c+) and plasmacytoid (CD123+) dendritic cells that have been activated with the cytokine thymic stromal lymphopoietin (TSLP). The presence of FoxP3 in naturally occurring Treg cells distinguishes them from other T cells. Mutations of the FOXP3 gene can prevent regulatory T cell development, causing the fatal autoimmune disease IPEX.

Treg-associated Disease, Disorder or Condition: As used herein, the term “Treg-associated Disease Disorder or Condition” refers to a disease, disorder or condition whose presence or severity correlates with a lack of Treg activity, and/or for which reduction in presence, severity or frequency correlates with elevated Treg activity.

pTreg stimulatory agent: As used herein, the term “pTreg stimulatory agent” refers to an agent characterized in that, when cells, tissues or organisms are exposed to the agent, the level and/or activity of pTregs is higher than under otherwise comparable conditions absent the agent.

### BRIEF DESCRIPTION OF THE DRAWING

FIG. 1 shows exemplary effects of short chain fatty acids produced by commensal bacteria stimulating in vitro generation of Treg cells: A) comparison of the effect of fecal extracts from SPF, antibiotic-treated (AVNM), or germ-free (GF) mice on in vitro induction of Foxp3 expression in naïve CD4+ T cells stimulated with CD3 antibody in the presence of Flt3L-elicited DC and TGF-β; B) HPLC fractionation of 2-nitrophenylhydrazine-HCl derivatized short chain fatty acids present in indicated fecal extracts; C) a comparison of the effect of indicated purified short chain fatty acids on in vitro induction of Foxp3 expression in naïve CD4+ T cells isolated from B6 or Foxp3GFP mice as described in (A); and D) a comparison of the effect of butyrate on Foxp3 induction in CNS1-sufficient and -deficient naïve CD4+ T cells from Foxp3GFP and Foxp3ΔCNS1 as described in (A).

FIG. 2 depicts how butyrate provision promotes extrathymic Treg cell generation in vivo: A), B) Flow cytometric analysis of Foxp3+ Treg cell subsets in the spleen and lymph nodes (LN) of AVNM-treated (AVNM) or untreated (SPF) B6 or Foxp3GFP mice treated with (+But; blue symbols) or without (black symbols) butyrate in drinking water; C) CNS1-deficient mice were treated with AVNM with or without butyrate as in (A) and analyzed for Foxp3 expression in splenic and lymph node (LN) CD4+ T cell populations; D) LC-MS analysis of butyrate in serum from CNS1-sufficient B6 (WT) and -deficient mice (CNS1) treated as in (A); E) Flow cytometric analysis of Foxp3+ Treg cell populations in colonic lamina propria of Foxp3GFP (left) and CNS1-deficient mice (right); F) Flow cytometric analysis of Foxp3 protein expression on a per cell basis in splenic Foxp3+ Treg cells in B6 mice treated with butyrate (+But) alone (SPF) or in combination with antibiotics (AVNM) as indicated and G) AVNM-treated Foxp3GFP (left) and CNS1-deficient mice (right) were administered acetate (Ace), propionate (Prop), butyrate (But), or no SCFA (AVNM) for a period of 3 weeks followed by analysis of Foxp3+ Treg cell subsets within CD4+ cells isolated from the colonic lamina propria (top panels) or spleens (bottom panels).

FIG. 3 depicts how butyrate acts within T cells to enhance acetylation of the Foxp3 locus and Foxp3 protein: A) induction of Foxp3 expression upon stimulation of naïve CD4+ T cells by CD3 antibody in the presence of butyrate-treated or untreated Flt3L-elicited DC and TGF-β; B) analysis of Foxp3 protein expression on a per-cell basis in Treg cells generated in the presence of butyrate pre-treated Flt3L-elicited DC [as in (a)]; C) percent of CD4+ cells expressing Foxp3 after 4 days in FACS-sorted naïve CD4+ T cells incubated with CD3/CD28 antibody-coated beads under Treg-inducing conditions; D) MFI of Foxp3 expression in Foxp3+CD4+ cells from (C); E) Thy1.1 expression in CD4+Foxp3+ splenocytes isolated from bi-cistronic Foxp3Thy1.1 reporter mice treated with AVNM with (+But) or without butyrate as described in FIG. 2A legend; F) CD4+Foxp3+ splenocytes from FoxpGFP reporter mice treated with AVNM with or without butyrate (as in FIG. 2a) were FACS-sorted and analyzed for Foxp3 mRNA expression by qPCR; G) AVI-tagged Foxp3-expressing TCli hybridoma cells were treated for 15 h with butyrate at the indicated concentrations followed by immunoprecipitation of tagged Foxp3 protein using streptavidin beads and immunoblotting for acetylated-lysine residues (top panel), total Foxp3 protein (middle panel) and tubulin (bottom panel) from pre-precipitation whole cell lysate; H) Analysis of suppressor capacity of GFP+ Treg cells sorted from antibiotic-treated (AVNM) Foxp3GFP mice administered (+But) or not administered butyrate in drinking water; and I) FACS-sorted naïve CD4+ T cells isolated from Foxp3GFP animals were incubated with CD3/CD28 antibody-coated beads under Treg-inducing conditions in the presence of indicated amounts of butyrate.

FIG. 4 depicts HDAC-inhibitory activity of butyrate decreases pro-inflammatory cytokine expression within DC to promote Treg induction: A) Histone acetylation in Flt3L-elicited DC from B6 mice treated with the indicated SCFA (500 μM) or TSA (10 nM) for 6 h followed by acid extraction of histones from isolated nuclei, SDS-PAGE and blotting with antibody for pan-acetylated H3. Total histone H3 served as a loading control; B) Induction of Foxp3 expression upon stimulation of naïve CD4+ T cells by CD3 antibody in the presence of SCFA or TSA, Flt3L-elicited DC and TGF-β; C) RelB gene expression quantified by qPCR in purified Flt3L-elicited DC from B6 mice treated for 6 h with SCFA or TSA, as in (A); and D) RelB gene expression quantified by qPCR in purified Flt3L-elicited DC from B6 mice treated with or without TSA in combination with, or in the absence of, butyrate at the indicated concentrations.

FIG. 5 depicts butyrate increasing the numbers of Treg cells in vivo: B6 mice were treated for one week with antibiotics (AVNM) in the absence or presence of butyrate (+But) in drinking water for two weeks.

FIG. 6 depicts how cytokine production by ex vivo isolated T effector cells is not increased in the presence of butyrate: A-D) B6 mice were treated with antibiotics (AVNM) in the absence or presence of butyrate (+But) in drinking water for 2 weeks. Cytokine production (IFN, IL-17, IL-13, IL-4) by splenic and lymph node Foxp3− CD4+ T cells was assessed by intracellular flow cytometry upon stimulation for 5 hours with CD3 and CD28 (5 μg mL-1 each) in the presence of Brefeldin A.

FIG. 7 shows that provision of butyrate to the colon via butyrate starch diet increases Tregs in colonic lamina propria: A), B) flow cytometric analysis of Foxp3+ Treg cell subsets isolated from the colonic lamina propria of mice fed ad libitum for 3 weeks with food formulated with control or butyrate starch.

FIG. 8 shows how pretreatment of splenic dendritic cell with butyrate is sufficient to increase total numbers of Treg cells and maintain dendritic cell viability during Treg induction: A) induction of Foxp3 expression upon stimulation of naïve CD4⁺ T cells by CD3 antibody in the presence of butyrate-treated or untreated Flt3L-elicited dendritic cells and TGF-β; B) FACS-sorted CD11c⁺MHCII^{hi} dendritic cells isolated from the spleen of unperturbed B6 mice were treated with butyrate at the indicated concentrations for 6 hours, washed and co-cultured with FACS purified naïve CD4+ T cells under Treg-inducing conditions; and C) as in (A), shown are the percent of surviving Flt3L-elicited CD11c+ dendritic cells in cultures as determined by live/dead staining and flow cytometry.

FIG. 9 shows GPCR sensing and butyrate transporters are not required for butyrate-dependent increase in Treg cell induction by dendritic cells: A) analysis of the ability of dendritic cells from Gpr109a^{+/+} (WT), Gpr109a^{+/-}, or Gpr109a^{-/-} (KO) mice to generate Treg cells in the presence of butyrate and B) dendritic cells were cultured for 30 minutes without or with pertussis toxin (Ptx) followed by addition of butyrate at the indicated concentrations for a total of 6 hours.

FIG. 10 shows butyrate and TSA treated dendritic cells exhibit similar gene induction profiles and act via redundant pathways: A) microarray expression analysis of dendritic cells treated for 6 hours with butyrate or TSA; B) data from LPS-stimulated dendritic cells were meta-analyzed and compared to dendritic cells treated with butyrate for 6 hours; C) cumulative distribution function plot of the fold expression of LPS response genes in butyrate treated dendritic cells for 6 hours over untreated control dendritic cells; and D) dendritic cells were pre-treated with trichostatin A (TSA) without or in combination with butyrate at the indicated concentrations for 6 hours, washed and co-cultured with FACS-purified naïve CD4+ T cells under Treg-inducing conditions. The data are shown as percent of CD4+ cells expressing Foxp3 after 4 days of culture. Data are representative of 2 independent experiments; error bars denote SEM.

FIG. 11 shows liposome encapsulated TSA delivered to dendritic cells increases the generation of Foxp3+ Tregs.

FIG. 12 shows that provision of butyrate to the colon via butyrate starch diet increases increase butyrate, but not propionate, concentrations; A)¹H NMR analysis of fecal pellets for butyrate concentration; B)¹H NMR analysis of fecal pellets for propionate concentration

### DETAILED DESCRIPTION OF CERTAIN EMBODIMENTS

The present invention provides, among other things, compositions and methods relating to inducing generation of regulatory T-cells (Treg) in a subject, for example who is suffering from or susceptible to a Treg-associated disease, disorder or condition. In part, the present invention is based on the surprising discovery that bacterial metabolites, in particular short chain fatty acids such as butyrate and propionate, can promote an increase in the number of Treg cells in extrathymic environments, and therefore act as pTreg stimulatory agents. The present invention provides technologies that achieve administration of such stimulatory agents to subjects. In some embodiments, such administration may be achieved through direct administration of the agents themselves. In some embodiments, such administration may be achieved through use of a therapeutic regimen that induces microbes present in a subject to produce or release them. In some embodiments, such a regimen may consist of or comprise administration of relevant microbes.

Intestinal microbes provide multicellular hosts with nutrients and confer resistance to infection. The delicate balance between pro- and anti-inflammatory mechanisms, essential for gut immune homeostasis, is affected by the composition of the commensal microbial community. Regulatory T (Treg) cells expressing transcription factor Foxp3 play a key role in limiting inflammatory responses in the intestine (Josefowicz, S. Z. et al. Nature, 2012, 482, 395-U1510). Although specific members of the commensal microbial community have been found to potentiate the generation of anti-inflammatory Treg or pro-inflammatory Th17 cells, the molecular cues driving this process remain elusive (Round, J. L. et al. PNAS, 2010, 107, 12204-12209; Ivanov, I I et al. Cell, 2009, 139, 485-98; Lathrop, S. K. et al. Nature, 2011, 478, 250-4; Atarashi, K. et al. Science, 2011, 331, 337-341; Atarashi, K. et al. Nature, 2013, 500, 232-6). Considering the vital metabolic function afforded by commensal microorganisms, it is reasonable to suspect their metabolic by-products are sensed by cells of the immune system and affect the balance between pro- and anti-inflammatory cells. In some embodiments, the present invention provides a short-chain fatty acid (SCFA), butyrate, produced by commensal microorganisms during starch fermentation, which facilitates extrathymic generation of Treg cells (or peripheral Tregs). In some embodiments, a boost in Treg cell numbers upon provision of butyrate is due to potentiation of extrathymic differentiation of Treg cells as the observed phenomenon was dependent upon intronic enhancer CNS1, essential for extrathymic, but dispensable for thymic Treg cell differentiation (Josefowicz, S. Z. et al. Nature, 2012, 482, 395-U1510; Zheng, Y. et al. Nature, 2010, 463, 808-12). In some embodiments, Treg cell generation in the periphery was also potentiated by propionate, another SCFA of microbial origin capable of HDAC inhibition. In some embodiments, acetate, another SCFA, lacks this activity. In some embodiments, bacterial metabolites mediate communication between the commensal microbiota and the immune system, affecting the balance between pro- and anti-inflammatory mechanisms.

Various aspects of the invention are described in detail in the following sections. The use of sections is not meant to limit the invention. Each section can apply to any aspect of the invention. In this application, the use of “or” means “and/or” unless stated otherwise.

### Regulatory T Cells

Regulatory T cells (Treg) are important in maintaining homeostasis, controlling the magnitude and duration of the inflammatory response, and in preventing autoimmune and allergic responses. There are two major classifications of Treg: natural Treg and peripheral Treg. Natural Treg, (nTreg) are a class of thymically generated T-cells while peripheral Treg (pTreg) develop in the periphery from naïve T cells in response to signals such as low doses of antigen, presence of certain microbes, lymphopenia or, in some cases, through activation by immature dendritic cells. In some cases, pTreg are thought to be generated in response to inflammatory conditions, particularly those which may be due at least in part to the absence of nTreg cells.

The Forkhead box P3 transcription factor (Foxp3) has been shown to be a key regulator in the differentiation and activity of Treg. In fact, loss-of-function mutations in the Foxp3 gene have been shown to lead to the lethal IPEX syndrome (immune dysregulation, polyendocrinopathy, enteropathy, X-linked). Patients with IPEX suffer from severe autoimmune responses, persistent eczema, and colitis.

In general Tregs are thought to be mainly involved in suppressing immune responses, functioning in part as a “self-check” for the immune system to prevent excessive reactions. In particular, Tregs are involved in maintaining tolerance to self-antigens, harmless agents such as pollen or food, and abrogating autoimmune disease.

Tregs are found throughout the body including, without limitation, the gut, skin, lung, and liver. Additionally, Treg cells may also be found in certain compartments of the body that are not directly exposed to the external environment such as the spleen, lymph nodes, and even adipose tissue. Each of these Treg cell populations is known or suspected to have one or more unique features and additional information may be found in Lehtimaki and Lahesmaa, 2013, Frontiers in Immunol., 4(294): 1-10, the disclosure of which is hereby incorporated in its entirety.

Typically, regulatory T cells are known to require TGF-β and IL-2 for proper activation and development. Blockade of TGF-β signaling has been shown to result in systemic inflammatory disease as a result of a deficiency of Treg and IL-2 knockout mice have been shown to fail to develop Treg. TGF-β may be particularly important, as it is known to stimulate Foxp3, the transcription factor that drives differentiation of T cells toward the Treg lineage.

Regulatory T cells are known to produce both IL-10 and TGF-β, both potent immune suppressive cytokines Additionally, Tregs are known to inhibit the ability of antigen presenting cells (APCs) to stimulate T cells. One proposed mechanism for APC inhibition is via CTLA-4, which is expressed by Foxp3⁺ Treg. It is thought that CTLA-4 may bind to B7 molecules on APCs and either block these molecules or remove them by causing internalization resulting in reduced availability of B7 and an inability to provide adequate co-stimulation for immune responses. Additional discussion regarding the origin, differentiation and function of Treg may be found in Dhamne et al., Peripheral and thymic Foxp3+ regulatory T cells in search of origin, distinction, and function, 2013, Frontiers in Immunol., 4 (253): 1-11, the disclosure of which is hereby incorporated in its entirety.

pTreg Stimulatory Agents

According to various embodiments, provided methods and compositions include one or more pTreg stimulating agents and/or strategies for reducing inflammation. As used herein a “pTreg stimulating agent” means a substance or method capable of stimulating (e.g., inducing) a subject's Treg cell population, particularly the peripheral regulatory T cells (pTregs) by conversion of naïve T cells (Tn cells) into regulatory T cells (pTregs).

In some embodiments, pTreg stimulating agents are biological agents, such as a histone deacetylase (HDAC) inhibitory agents. In some embodiments, pTreg stimulating agents are histone deacetylase (HDAC) inhibitory agents that acts to promote pTreg differentiation in a Foxp3-dependent manner.

Among other things, the present disclosure demonstrates that certain bacterial metabolites are able to induce pTreg generation. In some embodiments, bacterial metabolites induce pTreg differentiation in a Foxp3—dependent manner. In some embodiments, pTreg stimulating agents include histone deacetylase (HDAC) inhibitory agents which are bacterial metabolites. In some embodiments, pTreg stimulating agents are short chain fatty acids. In some embodiments, pTreg stimulating agents are short chain fatty acids that include but are not limited to: butyrate, propionate, succinate, formate, valproate, phenylbutyrate, L-lactate, 2-ethylbutyrate, isovalerate, isobutyric acid, valeric acid and analogs thereof.

In some embodiments, pTreg stimulatory agents produced by bacteria are administered by inducing the local bacteria to produce and/or release pTreg stimulating agents. In some embodiments, the bacteria that produce pTreg stimulatory agents are themselves administered to induce pTreg generation. In some embodiments, the bacteria that produce pTreg stimulatory agents are administered in a probiotic form. In some embodiments, the bacteria that produce pTreg stimulatory agents are administered to a subject orally. In some embodiments, bacteria that produce pTreg stimulatory agents are administered to a subject topically, or by enema delivery intrarectally.

Identification and/or Characterization of pTreg Stimulatory Agents

In some embodiments, one or more tests are performed to verify and/or quantitate the degree of pTreg generation. In some embodiments, generation of pTregs may be verified and/or quantified through detection of increased numbers of Foxp3 cells. In some embodiments, generation of pTreg may be verified and/or quantified through detection of a increased number of Foxp3⁺ CD25⁺ CD4⁺ cells

In some embodiments, the generation of pTreg results in at least one symptom or feature of inflammation being reduced in intensity, severity, duration, or frequency, and/or has delayed in onset.

In some embodiments, the present invention provides methods and systems for identifying and/or characterizing pTreg stimulating agents and/or protocols. In some embodiments, provided methods and systems include administering one or more candidate pTreg stimulating agents and/or protocols to a population of T cells and assaying for proliferation. In some embodiments, provided methods and systems include administering one or more candidate pTreg stimulating agents and/or protocols to a population of dendritic cells and assaying for pTreg proliferation. In some embodiments, the population of pTregs is an in vitro population. In some embodiments, the pTreg population is an in vivo population. In some embodiments, a candidate pTreg stimulating agent and/or protocol is considered a pTreg stimulating agent and/or protocol if administration results in a increase in Treg population by 10%, 20%, 30%, 40%, 50%, 60%, 70%, 80%, 90%, or more as compared to a similar pTreg population that was not exposed to the agent(s) and/or protocol(s).

### Inflammation

Inflammation, as used herein, refers to the localized protective response of vascular tissues to injury, irritation or infection. Inflammatory conditions are characterized by one or more of the following symptoms: redness, swelling, pain and loss of function. Inflammation is a protective attempt by the organism to remove the harmful stimuli and begin the healing process. Although infection is caused by a microorganism, inflammation is one of the responses of the organism to the pathogen.

Inflammation can be classified as either acute or chronic. Acute inflammation is the initial response of the body to harmful stimuli and is achieved by the increased movement of plasma and leukocytes (especially granulocytes) from the blood into the injured tissues. A cascade of biochemical events propagates and matures the inflammatory response, involving the local vascular system, the immune system, and various cells within the injured tissue. Prolonged inflammation, known as chronic inflammation, leads to a progressive shift in the type of cells present at the site of inflammation and is characterized by simultaneous destruction and healing of the tissue from the inflammatory process.

Inflammation may be caused by a number of agents, including infectious pathogens, toxins, chemical irritants, physical injury, hypersensitive immune reactions, radiation, foreign irritants (dirt, debris, etc.), frostbite, and burns. Types of inflammation include colitis, bursitis, appendicitis, dermatitis, cystitis, rhinitis, tendonitis, tonsillitis, vasculitis, and phlebitis. In some embodiments, inflammatory conditions are Treg-associated Diseases Disorders and Conditions.

Therapeutic Uses of pTreg Stimulatory Agents

It is contemplated that provided methods and compositions may be used to treat any of a variety of Treg-associated Diseases Disorders and Conditions. In some embodiments, the Treg-associated Diseases Disorders and Conditions treated by the provided invention include colitis, asthma, chronic peptic ulcer, tuberculosis, rheumatoid arthritis, chronic periodontitis, Crohn's disease, chronic sinusitis, pelvic inflammatory disease, hepatitis, inflammatory bowel disease, sarcoidosis, vasculitis, celiac disease, autoimmune disease, reperfusion injury, transplant rejection, and infection.

Therapeutic uses include administration of pTreg stimulatory agents alone or in combination with other treatments, including more than one type of pTreg stimulatory agent. In some embodiments, therapeutic uses include administration of pTreg stimulatory agents including HDAC inhibitory agents, bacterial metabolites, and short chain fatty acids. In some embodiments, therapeutic uses include administration of pTreg stimulatory agents in combination with other compositions, including but not limited to other anti-inflammatory compositions. In some embodiments, therapeutic uses include administration of bacteria that are induced to produce pTreg stimulatory agents. In some embodiments, therapeutic uses include administration of bacteria that produce pTreg stimulatory agents.

Therapeutic uses include administration of pTreg stimulatory agents via routes selected from intradermal, intramuscular, intraoperative, intrathecal, intravenous, nasal, ocular, oral, parental, rectal, subcutaneous, topical, and transdermal. In some embodiments, therapeutic use includes oral and/or topical administration of bacteria that are induced to produce Treg stimulatory agents.

### Pharmaceutical Compositions

In some embodiments, the present invention provides pharmaceutical compositions comprising one or more provided Treg stimulating agent together with one or more pharmaceutically acceptable excipients.

In some embodiments, provided pharmaceutical compositions may be prepared by any appropriate method, for example as known or hereafter developed in the art of pharmacology. In general, such preparatory methods include the step of bringing a provided Treg stimulating agent into association with one or more pharmaceutically acceptable excipients, and then, if necessary and/or desirable, shaping and/or packaging the product into an appropriate form for administration, for example as or in a single- or multi-dose unit.

In some embodiments, compositions may be prepared, packaged, and/or sold in bulk, as a single unit dose, and/or as a plurality of single unit doses. As used herein, a “unit dose” is a discrete amount of the pharmaceutical composition comprising a predetermined amount of one or more provided Treg stimulating agent. The amount of the provided Treg stimulating agent is generally equal to the dosage of the provided Treg stimulating agent which would be administered to a subject and/or a convenient fraction of such a dosage such as, for example, one-half or one-third of such a dosage.

In some embodiments, provided pharmaceutical compositions are specifically formulated for mucosal delivery (e.g., oral, nasal, rectal or sublingual delivery). In some embodiments, pharmaceutical compositions are specifically formulated for oral delivery as being conjugated to starch and mixed with food.

In some embodiments, appropriate excipients for use in provided pharmaceutical compositions may, for example, include one or more pharmaceutically acceptable solvents, dispersion media, granulating media, diluents, or other liquid vehicles, dispersion or suspension aids, surface active agents and/or emulsifiers, isotonic agents, thickening or emulsifying agents, preservatives, solid binders, lubricants, disintegrating agents, binding agents, preservatives, buffering agents and the like, as suited to the particular dosage form desired. Alternatively or additionally, pharmaceutically acceptable excipients such as cocoa butter and/or suppository waxes, coloring agents, coating agents, sweetening, flavoring, and/or perfuming agents can be utilized. Remington's The Science and Practice of Pharmacy, 21^{st} Edition, A. R. Gennaro (Lippincott, Williams & Wilkins, Baltimore, Md., 2005; incorporated herein by reference) discloses various excipients used in formulating pharmaceutical compositions and known techniques for the preparation thereof.

In some embodiments, an appropriate excipient is at least 95%, at least 96%, at least 97%, at least 98%, at least 99%, or 100% pure. In some embodiments, an excipient is approved by United States Food and Drug Administration. In some embodiments, an excipient is pharmaceutical grade. In some embodiments, an excipient meets the standards of the United States Pharmacopoeia (USP), the European Pharmacopoeia (EP), the British Pharmacopoeia, and/or other International Pharmacopoeia.

In some embodiments, liquid dosage forms (e.g., for oral and/or parenteral administration) include, but are not limited to, emulsions, microemulsions, solutions, suspensions, syrups, and/or elixirs. In addition to provided Treg stimulating agent(s), liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can include adjuvants such as wetting agents, emulsifying and suspending agents, sweetening, flavoring, and/or perfuming agents. In certain embodiments for parenteral administration, compositions are mixed with solubilizing agents such a CREMOPHOR®, alcohols, oils, modified oils, glycols, polysorbates, cyclodextrins, polymers, and/or combinations thereof.

In some embodiments, injectable preparations, for example, sterile aqueous or oleaginous suspensions, may be formulated according to known methods using suitable dispersing agents, wetting agents, and/or suspending agents. Sterile liquid preparations may be, for example, solutions, suspensions, and/or emulsions in nontoxic parenterally acceptable diluents and/or solvents, for example, as a solution in 1,3-butanediol. Among the acceptable vehicles and solvents that may be employed, for example, are water, Ringer's solution, U.S.P., and isotonic sodium chloride solution. Sterile, fixed oils are conventionally employed as a solvent or suspending medium. For this purpose any bland fixed oil can be employed including synthetic mono- or diglycerides. Fatty acids such as oleic acid can be used in the preparation of liquid formulations.

Liquid formulations can be sterilized, for example, by filtration through a bacterial-retaining filter, and/or by incorporating sterilizing agents in the form of sterile solid compositions which can be dissolved or dispersed in sterile water or other sterile injectable medium prior to use.

In some embodiments, one or more strategies may be utilized prolong and/or delay the effect of a provided Treg stimulating agent after delivery.

In some embodiments, provided pharmaceutical compositions may be formulated as suppositories, for example for rectal or vaginal delivery. In some embodiments, suppository formulations can be prepared by mixing utilizing suitable non-irritating excipients such as cocoa butter, polyethylene glycol or a suppository wax which are solid at ambient temperature but liquid at body temperature and therefore melt in the body (e.g., in the rectum or vaginal cavity) and release the provided Treg stimulating agent.

In some embodiments, solid dosage forms (e.g., for oral administration) include capsules, tablets, pills, powders, and/or granules. In such solid dosage forms, the provided Treg stimulating agent(s) may be mixed with at least one inert, pharmaceutically acceptable excipient such as sodium citrate or dicalcium phosphate and/or fillers or extenders (e.g., starches, lactose, sucrose, glucose, mannitol, and silicic acid), binders (e.g., carboxymethylcellulose, alginates, gelatin, polyvinylpyrrolidinone, sucrose, and acacia), humectants (e.g., glycerol), disintegrating agents (e.g., agar, calcium carbonate, potato starch, tapioca starch, alginic acid, certain silicates, and sodium carbonate), solution retarding agents (e.g., paraffin), absorption accelerators (e.g., quaternary ammonium compounds), wetting agents (e.g., cetyl alcohol and glycerol monostearate), absorbents (e.g., kaolin and bentonite clay), and lubricants (e.g., talc, calcium stearate, magnesium stearate, solid polyethylene glycols, sodium lauryl sulfate), and mixtures thereof. In the case of capsules, tablets and pills, the dosage form may comprise buffering agents.

In some embodiments, solid compositions of a similar type may be employed as fillers in soft and/or hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art.

Exemplary enteric coatings include, but are not limited to, one or more of the following: cellulose acetate phthalate; methyl acrylate-methacrylic acid copolymers; cellulose acetate succinate; hydroxy propyl methyl cellulose phthalate; hydroxy propyl methyl cellulose acetate succinate (hypromellose acetate succinate); HP55; polyvinyl acetate phthalate (PVAP); methyl methacrylate-methacrylic acid copolymers; methacrylic acid copolymers, cellulose acetate (and its succinate and phthalate version); styrol maleic acid co-polymers; polymethacrylic acid/acrylic acid copolymer; hydroxyethyl ethyl cellulose phthalate; hydroxypropyl methyl cellulose acetate succinate; cellulose acetate tetrahydrophtalate; acrylic resin; shellac, and combinations thereof.

In some embodiments, solid dosage forms may optionally comprise opacifying agents and can be of a composition that they release the provided Treg stimulating agent(s) only, or preferentially, in a certain part of the intestinal tract, optionally, in a delayed manner Examples of embedding compositions which can be used include polymeric substances and waxes. Solid compositions of a similar type may be employed as fillers in soft and hard-filled gelatin capsules using such excipients as lactose or milk sugar as well as high molecular weight polyethylene glycols and the like.

In some embodiments, the present invention provides compositions for topical and/or transdermal delivery, e.g., as a cream, liniment, ointment, oil, foam, spray, lotion, liquid, powder, thickening lotion, or gel. Particular exemplary such formulations may be prepared, for example, as products such as skin softeners, nutritional lotion type emulsions, cleansing lotions, cleansing creams, skin milks, emollient lotions, massage creams, emollient creams, make-up bases, lipsticks, facial packs or facial gels, cleaner formulations such as shampoos, rinses, body cleansers, hair-tonics, or soaps, or dermatological compositions such as lotions, ointments, gels, creams, liniments, patches, deodorants, or sprays.

In some embodiments, provided compositions are stable for extended periods of time, such as 1 week, 2 weeks, 1 month, 2 months, 6 months, 1 year, 2 years, 3 years, or more. In some embodiments, provided compositions are easily transportable and may even be sent via traditional courier or other package delivery service. Accordingly, some embodiments may be useful in situations of disease outbreak, such as epidemics, or attacks with biological agents at least in part due to their ability to be stored for long periods of time and transported quickly, easily, and safely. Such attributes may allow for rapid distribution of provided compositions to those in need.

In some embodiments, it may be advantageous to release Treg stimulating agent(s), at various locations along a subject's gastrointestinal (GI) tract. In some embodiments, it may be advantageous to release Treg stimulating agent(s), for example, an agent, in a subject's mouth as well as one or more locations along the subject's GI tract. In some embodiments, it may be advantageous to release Treg stimulating agent(s), for example, an agent, in a subject's GI tract, including but not limited to the stomach, intestines, and colon. Accordingly, in some embodiments, a plurality of provided compositions (e.g., two or more) may be administered to a single subject to facilitate release of Treg stimulating agent(s) at multiple locations. In some embodiments, each of the plurality of compositions has a different release profile, such as provided by various enteric coatings, for example. In some embodiments, each of the plurality of compositions has a similar release profile. In some embodiments, the plurality of compositions comprises one or more Treg stimulating agents. In some embodiments, each of the plurality of administered compositions comprises a different Treg stimulating agent. In some embodiments, each of the plurality of compositions comprises the same Treg stimulating agent.

### Dosing

It is contemplated that a variety of dosing regimen may be used in accordance with various embodiments. In some embodiments, the step of stimulating comprises administering at least two doses of a Treg stimulating agent, separated by a period of time. In some embodiments, the step of stimulating comprises administering at least three, four, five, six or more than six doses of a Treg stimulating agent, each separated by a period of time. In some embodiments, the period of time between each administration is the same. In some embodiments, the period of time between each administration is different. In some embodiments, the period of time between doses may be 1 minute, 15 minutes, 30 minutes, 1 hour, 2 hours, 3 hours, 6 hours, 12 hours, 24 hours, 48 hours, 72 hours, 96 hours, 1 week, 2 weeks, or 1 month. In some embodiments, the period of time between doses is greater than 1 month. In some embodiments, each dose is administered substantially simultaneously (e.g., sequentially).

According to various embodiments comprising administration of two or more doses of a Treg stimulating agent, the dose of Treg stimulating agent may vary according to sound medical judgment. In some embodiments, each dose of a Treg stimulating agent is the same. In some embodiments, each dose of a Treg stimulating agent may vary from one or more other doses.

In some embodiments, a Treg stimulating agent is administered at a dose equal to or approximating a therapeutically effective amount. In some embodiments, a therapeutically effective amount of a Treg stimulating agent may be an amount ranging from about 0.001 to about 1,000 mg/kg. In some embodiments, a therapeutically effective amount may be, for example, about 0.001 to 500 mg/kg weight, e.g., from about 0.001 to 400 mg/kg weight, from about 0.001 to 300 mg/kg weight, from about 0.001 to 200 mg/kg weight, from about 0.001 to 100 mg/kg weight, from about 0.001 to 90 mg/kg weight, from about 0.001 to 80 mg/kg weight, from about 0.001 to 70 mg/kg weight, from about 0.001 to 60 mg/kg weight, from about 0.001 to 50 mg/kg weight, from about 0.001 to 40 mg/kg weight, from about 0.001 to 30 mg/kg weight, from about 0.001 to 25 mg/kg weight, from about 0.001 to 20 mg/kg weight, from about 0.001 to 15 mg/kg weight, from about 0.001 to 10 mg/kg weight. In some embodiments, the therapeutically effective amount described herein is provided in one dose. In some embodiments, the therapeutically effective amount described herein is provided in one day.

In some embodiments, a therapeutically effective dosage amount may be, for example, about 0.0001 to about 0.1 mg/kg weight, e.g. from about 0.0001 to 0.09 mg/kg weight, from about 0.0001 to 0.08 mg/kg weight, from about 0.0001 to 0.07 mg/kg weight, from about 0.0001 to 0.06 mg/kg weight, from about 0.0001 to 0.05 mg/kg weight, from about 0.0001 to about 0.04 mg/kg weight, from about 0.0001 to 0.03 mg/kg weight, from about 0.0001 to 0.02 mg/kg weight, from about 0.0001 to 0.019 mg/kg weight, from about 0.0001 to 0.018 mg/kg weight, from about 0.0001 to 0.017 mg/kg weight, from about 0.0001 to 0.016 mg/kg weight, from about 0.0001 to 0.015 mg/kg weight, from about 0.0001 to 0.014 mg/kg weight, from about 0.0001 to 0.013 mg/kg weight, from about 0.0001 to 0.012 mg/kg weight, from about 0.0001 to 0.011 mg/kg weight, from about 0.0001 to 0.01 mg/kg weight, from about 0.0001 to 0.009 mg/kg weight, from about 0.0001 to 0.008 mg/kg weight, from about 0.0001 to 0.007 mg/kg weight, from about 0.0001 to 0.006 mg/kg weight, from about 0.0001 to 0.005 mg/kg weight, from about 0.0001 to 0.004 mg/kg weight, from about 0.0001 to 0.003 mg/kg weight, from about 0.0001 to 0.002 mg/kg weight. The effective dose for a particular individual can be varied (e.g., increased or decreased) over time, depending on the needs of the individual.

### Routes of Administration

In some embodiments, provided Treg stimulating agents and compositions comprising the same may be formulated for any appropriate route of delivery. In some embodiments, provided Treg stimulating agents and compositions comprising the same may be formulated for any route of delivery, including, but not limited to, bronchial instillation, and/or inhalation; buccal, enteral, interdermal, intra-arterial (IA), intradermal, intragastric (IG), intramedullary, intramuscular (IM), intranasal, intraperitoneal (IP), intrathecal, intratracheal instillation (by), intravenous (IV), intraventricular, mucosal, nasal spray, and/or aerosol, oral (PO), as an oral spray, rectal (PR), subcutaneous (SQ), sublingual; topical and/or transdermal (e.g., by lotions, creams, liniments, ointments, powders, gels, drops, etc.), transdermal, vaginal, vitreal, and/or through a portal vein catheter; and/or combinations thereof. In some embodiments, the present invention provides methods of administration of Treg stimulating agents and compositions comprising the same via mucosal administration. In some embodiments, the present invention provides methods of administration of Treg stimulating agents and compositions comprising the same via oral administration. In some embodiments, provided Treg stimulating agents and compositions comprising the same may be formulated as a probiotic for oral delivery. In some embodiments, provided Treg stimulating agents and compositions comprising the same may be formulated as a probiotic for topical delivery. In some embodiments, provided Treg stimulating agents and compositions comprising the same may be administered orally as bacteria that produce pTreg stimulating agents. In some embodiments, provided Treg stimulating agents and compositions comprising the same may be administered topically as bacteria that produce pTreg stimulating agents.

### Kits

In some embodiments, the present invention further provides kits or other articles of manufacture which contain one or more Treg stimulating agents or formulations containing the same, and provides instructions for its reconstitution (if lyophilized) and/or use. In some embodiments, a kit may comprise (i) at least one provided Treg stimulating agent or composition comprising the same; and (ii) at least one pharmaceutically acceptable excipient; and, optionally, (iii) instructions for use.

Kits or other articles of manufacture may include a container, a syringe, vial and any other articles, devices or equipment useful in administration (e.g., subcutaneous, by inhalation). Suitable containers include, for example, bottles, vials, syringes (e.g., pre-filled syringes), ampules, cartridges, reservoirs, or lyo-jects. The container may be formed from a variety of materials such as glass or plastic. In some embodiments, a container is a pre-filled syringe. Suitable pre-filled syringes include, but are not limited to, borosilicate glass syringes with baked silicone coating, borosilicate glass syringes with sprayed silicone, or plastic resin syringes without silicone.

Typically, the container may holds formulations and a label on, or associated with, the container that may indicate directions for reconstitution and/or use. For example, the label may indicate that the formulation is reconstituted to concentrations as described above. The label may further indicate that the formulation is useful or intended for, for example, subcutaneous administration. In some embodiments, a container may contain a single dose of a stable formulation containing one or more Treg stimulating agents. In various embodiments, a single dose of the stable formulation is present in a volume of less than about 15 ml, 10 ml, 5.0 ml, 4.0 ml, 3.5 ml, 3.0 ml, 2.5 ml, 2.0 ml, 1.5 ml, 1.0 ml, or 0.5 ml. Alternatively, a container holding the formulation may be a multi-use vial, which allows for repeat administrations (e.g., from 2-6 administrations) of the formulation. Kits or other articles of manufacture may further include a second container comprising a suitable diluent (e.g., BWFL saline, buffered saline). Upon mixing of the diluent and the formulation, the final protein concentration in the reconstituted formulation will generally be at least 1 mg/ml (e.g., at least 5 mg/ml, at least 10 mg/ml, at least 20 mg/ml, at least 30 mg/ml, at least 40 mg/ml, at least 50 mg/ml, at least 75 mg/ml, at least 100 mg/ml). Kits or other articles of manufacture may further include other materials desirable from a commercial and user standpoint, including other buffers, diluents, filters, needles, syringes, and package inserts with instructions for use. In some embodiments, kits or other articles of manufacture may include an instruction for self-administration.

In some embodiments, kits include multiple (e.g., 1, 2, 3, 4, 5, 6, 7, 8, 9, 10, 11, 12, 13, 14, 15, 16, 17, 18, 19, 20, or more) doses of provided Treg stimulating agents and/or compositions comprising the same. In some embodiments, kits include multiple (e.g., 1, 2, 3, 4, 5, 6, 7, 8, 9, 10, 11, 12, 13, 14, 15, 16, 17, 18, 19, 20, or more) populations of provided Treg stimulating agents and/or compositions comprising the same having different functional elements (e.g., Treg stimulating agents). In some embodiments, multiple populations of provided Treg stimulating agents and/or compositions comprising the same are packaged separately from one another in provided kits. In some embodiments, provided kits may include provided compositions and one or more other therapeutic agents intended for administration with the provided compositions.

### Combination Therapy

In some embodiments, provided Treg stimulating agents and compositions comprising the same are combined with other therapies to treat a Treg-associated Disease Disorder or Condition. In some embodiments, provided Treg stimulating agents and compositions comprising the same are combined with forms of treatment including but not limited to pharmacotherapy, chemotherapy, mesotherapy, medical devices, surgery, gene therapy, hormone therapy, radiotherapy, phototherapy, electrotherapy, thermotherapy, and cryotherapy. In some embodiments, provided Treg stimulating agents and compositions comprising the same are combined with biologics, cells, proteins, steroids, hormones, cytokines, enzymes, peptides, polypeptides, amino acids, nucleic acids, DNA, RNA, mRNA, tRNA, siRNA, dsRNA, DNA vaccines, antibodies, monoclonal antibodies, polyclonal antibodies, antibody-drug conjugates, antivirals, antibiotics, antifungals and any conjugates thereof.

In some embodiments, provided Treg stimulating agents and compositions comprising the same are combined with anti-inflammatory agents to treat pTreg-associated Diseases Disorders or Conditions. Anti-inflammatory agents include both steroids and non-steroidal anti-inflammatory drugs (NSAID). In some embodiments, provided Treg stimulating agents and compositions comprising the same are combined with steroids, including but not limited to glucocorticoids and corticosteroids. In some embodiments, provided Treg stimulating agents and compositions comprising the same are combined with non-steroidal anti-inflammatory drugs, including but not limited to ibuprofen, aspirin, naproxen sodium, celecoxib, sulindac, oxaprozin, salsalate, diflunisal, piroxicam, indomethacin, etodolac, meloxicam, naproxen, nabumetone, tromethamine, diclofenac, esomeprazole, and acetaminophen.

In some embodiments, provided Treg stimulating agents and compositions comprising the same are combined with medical imaging modalities to treat and monitor Treg-associated Diseases Disorders or Conditions. In some embodiments, provided Treg stimulating agents and compositions comprising the same are combined with medical imaging modalities including but not limited to echocardiography, thermography, tomography, photoacoustic imaging, ultrasound, magnetic resonance imaging, nuclear medicine, elastography, positron emission tomography, computed tomography, and fluorescence tomography.

### EXAMPLES

### Example 1

### Materials and Methods

Unless otherwise specified, the methods used in Examples 2-11 are as follows:

### Mice

Foxp3ΔCNS1 (CNS1 knockout), Foxp3GFP, Foxp3Thy1.1 and Gpr109a−/− mice were used in the experiments as previously described (Zheng. Y. et al. Nature, 2010, 463, 808-12; Fontenot, J. D. et al. Immunity, 2005, 22, 329-41; Kim, J. M., et al. Nat Immunol, 2007, 8, 191-7). Male C57BL/6 (B6) mice were purchased from the Jackson Laboratory and groups of 5 co-housed mice were randomly assigned to treatment vs. control groups after confirmation that age and weight were in accordance between groups. Male mice were used for all experiments. All strains were maintained in the Sloan-Kettering Institute animal facility in accordance with institutional guidelines. Mice were sacrificed by CO2 asphyxiation then blindly processed for tissue harvest thereafter. For antibiotic treatment, mice at 5-6 weeks of age were treated with 1 g L-1 metronidazole (Sigma-Aldrich), 0.5 g L-1 vancomycin (Hospira), 1 g L-1 ampicillin (Sigma-Aldrich) and 1 g L-1 kanamycin (Fisher Scientific) dissolved in drinking water. For butyrate, acetate and propionate administration, each SCFA was added to drinking water containing antibiotics (as described above) at a final concentration of 36 mM and pH-adjusted, if needed, to match that of antibiotic-only water. Butyrate was administered to mice after prior treatment with antibiotics for at least 1 week.

### Cell Isolation and FACS Staining

For in vitro experiments, CD4+ T cells and CD11c+ dendritic cells were enriched using mouse CD4 (L3T4, Invitrogen) and mouse CD11c (N418, BioLegend) antibodies, respectively, that were biotinylated for use with streptavidin-Dynabeads (Invitrogen). Enriched cells were then sorted on a FACSAria II cell sorter (BD Biosciences) for in vitro assays. Intracellular staining for IL-17, IFN-γ, IL-4, IL-13 and Foxp3 was performed using the Foxp3 staining kit (eBiosciences). Cytokine staining was performed after re-stimulation with CD3 antibody and CD28 antibody (5 μg ml-1 each) in the presence of Golgi-plug (BD Biosciences) for 5 hours.

### Dendritic Cell Generation and Isolation

DC were expanded in vivo by subcutaneous injection of B16 melanoma cells secreting FLT3-ligand into the left hind flank of mice as indicated. Once tumors were visible, spleens from injected animals were dissociated in RPMI 1640 medium containing 1.67 U mL-1 liberase TL (Roche) and 50 μg mL-1 DNAse I (Roche) for 20 min at 37° C. with shaking EDTA was then added at a final concentration of 5 mM to stop digestion and the resulting homogenate was processed for CD11c+ cell isolation using the MACS mouse CD11c (N418) purification kit (Miltenyi Biotec).

### In Vitro Assays

In vitro Foxp3 induction assays were performed by co-culturing DC with 5.5×104 CD4+CD44loCD62LhiCD25-naïve T cells in the presence of 1 μg ml-1 of CD3 antibody, 1 ng mL-1 TGF-β, and 100 U ml-1 IL-2, in 96-well flat-bottom plates for 4 d. For Foxp3 induction in the presence of butyrate- or TSA-pretreated DC, TGF-β was used at 0.1 ng mL-1 final concentration. In vitro induction assays in the absence of DC were performed by incubating 5.5×104 naïve CD4+ T cells with 1 ng mL-1 TGF-β, 100 U ml-1 IL-2, and a 1:1 cell-to-bead ratio of CD3/CD28 T activator Dynabeads (Invitrogen). For in vitro suppression assays, 4×104 naïve CD4+ T cells were FACS-sorted from B6 mice and cultured with graded numbers of CD4+Foxp3+ Treg cells FACS-sorted from Foxp3GFP mice treated with antibiotics and with or without butyrate, in the presence of 105 irradiated T cell-depleted splenocytes and 1 μg ml-1 CD3 antibody in a 96-well round-bottom plate for 80 h. Proliferation of T cells was assessed by [3H]-thymidine incorporation during the final 8 h of culture.

### Chromatin Immunoprecipitation (ChIP)-qPCR Assays

H3K27Ac ChIP-qPCR was performed as previously described (Samstein, R. M. et al. Cell, 2012, 151, 153-66). Briefly, fixed cells were lysed and mono- and poly-nucleosomes were obtained by partial digestion with micrococcal nuclease (12,000 U mL-1) in 1 min at 37° C. EDTA was added to a final concentration of 50 mM to stop the reaction, and digested nuclei were resuspended in nuclear lysis buffer with 1% SDS. After sonication, 1 ug H3K27Ac-specific antibody (Abcam, ab4729) was used to precipitate H3K27Ac-bound chromatin. Washing and de-crosslinking was performed as described (Samstein, R. M. et al. Cell, 2012, 151, 153-66).

### Stool Sample Collection

Stool samples were collected directly into sterile tubes from live mice and snap-frozen before preparation of material for SCFA quantification by HPLC or LC-MS (see corresponding section for further sample processing).

### HPLC Assays

HPLC analysis was performed for analysis of derivatized stool extracts as previously described (Torii, T. et al. Ann. Clin. Biochem, 2010, 47, 447-452). Briefly, flash-frozen stool samples were extracted with 70% ethanol and brought to a final concentration of 0.1 μg μL-1. Debris was removed by centrifugation and 300 μL of supernatant was transferred to a new tube and combined with 50 μL of internal standard (2-ethylbutyric acid, 200 mM in 50% aqueous methanol), 300 μL of dehydrated pyridine 3% v/v (Wako) in ethanol, 300 μL of 250 mM N-(3-dimethlaminopropyl)-N′-ethylcarbodiimide hydrochloride (Sigma-Aldrich) in ethanol, and 300 μL of 20 mM 2-nitrophenylhydrazine hydrochloride (Tokyo Chemical) in ethanol. Samples were incubated at 60° C. for 20 min and 200 μL of potassium hydroxide 15% w/v dissolved 80/20 in methanol was added to stop the derivatization reaction. Samples were incubated again at 60° C. for 20 min and transferred into a glass conical tube containing 3 mL of 0.5 M phosphoric acid. The organic phase was extracted by shaking with 4 mL diethyl ether and transferred to a new glass conical containing water to extract any remaining aqueous compounds. The organic phase containing the derivatized SCFA was transferred into a new 5 mL glass vial and evaporated overnight in a fume hood. Derivatized SCFA were resuspended in 100 μL of mobile phase (below) and 20 μL was chromatographed on a Shimadzu HPLC system equipped with a Vydac 2.1×30 mm 300 A C18 column run at 200 μL min-1 in methanol/acetonitrile/TFA (30%/16%/0.1% v/v) and monitored for absorbance at 400 nm.

### LC-MS Assays

300 μl 80% methanol containing deuterated short chain fatty acid internal standards (Cambridge Isotope Laboratories) was added to 70 μL serum and incubated at −80° C. for 30 min. Samples were then centrifuged at 4° C. at 14000 rpm for 15 min to precipitate protein. Pure short chain fatty acid standards (Sigma-Aldrich) were also prepared in 300 μl 80% methanol containing internal standards to produce a calibration curve from 0.25 μM to 50 μM. 80% methanol extracts were combined with 300 μl 250 mM N-(3-dimethylaminopropyl)-N′-ethylcarbodiimide hydrochloride in ethanol, 300 μL 20 mM 2-nitrophenylhydrazine hydrochloride in ethanol and 300 μL 3% pyridine in ethanol in a glass tube and reacted at 60° C. for 20 min. The reaction was quenched with 200 μL potassium hydroxide solution (15% KOH:MeOH, 8:2 v/v) at 60° C. for 20 min. After cooling, the mixture was adjusted to pH 4 with 0.25 M HCl. Derivatized short chain fatty acids were then extracted with 4 mL ether and washed with 4 mL water before drying under a nitrogen stream. The dried sample was dissolved in 150 μL methanol, and 5 μL was injected for LC-MS analysis.

### Butyrate Enemas

Mice were anesthetized with isoflurane and injected intrarectally with 200 μL of 50 mM butyric acid (pH 4.0) or pH-matched water delivered through a 1.2 mm-diameter polyurethane catheter (Access Technologies, Skokie, Ill.). Enemas were administered for 7 days.

### Butyrate Starch Chow

High-amylose maize starch was formulated for conjugation of butyrate molecules to the starch particle, or control cooked in the absence of butyrate as the control starch. These additives were obtained from National Starch under the name StraPlus Hylon VII (control) or StarPlus Butyrate Starch (butyrate conjugated to Hylon VII) and added at 15% to the TestDiet AIN-93G in place of cornstarch. For further details see Bajka et al. Nutrition Research. Volume 30, Issue 6, June 2010, Pages 427-434. Animals were fed these diets for a period of three weeks prior to assessment of increases in Treg cells in the colon.

### TSA Liposome Encapsulation

TSA was encapsulated in lipid particles containing mixtures of lipids for release of contents only upon entry into cells. For details see Karve et al. Langmuir 2008, 24, 5679-5688. To monitor incorporation of TSA into liposomes, lipids were dissociated with hydrogenated Triton-X and monitored for increased UV absorption compared to free TSA and ‘empty’ liposomes. >85% of free TSA is incorporated into the lipid particles.

### Example 2

### SCFA Produced by Commensal Bacteria Stimulate In Vitro Generation of Treg Cells

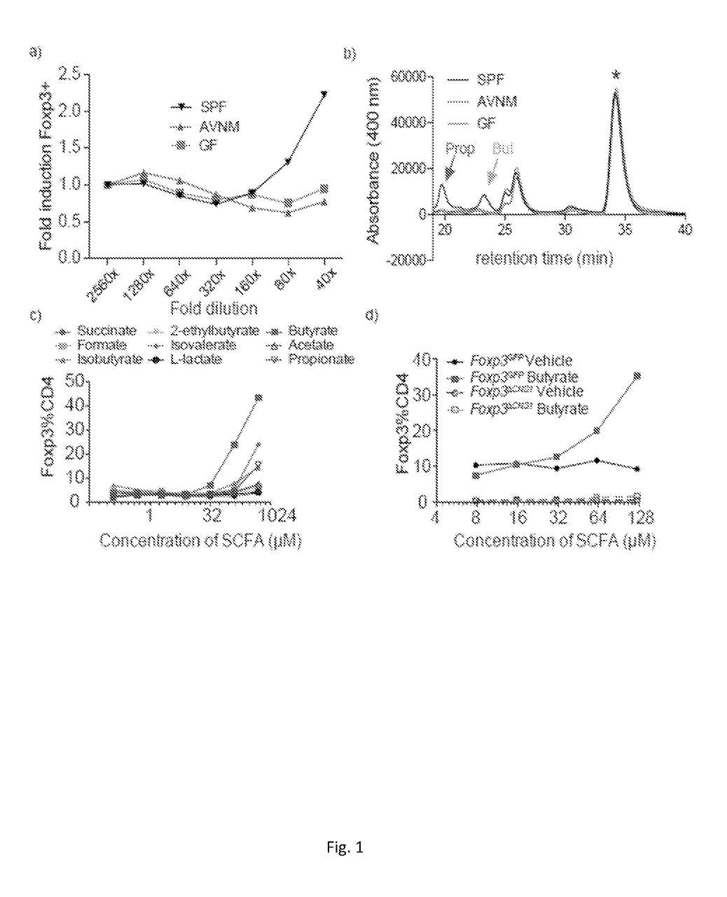

If microbial metabolites facilitate generation of extrathymic Treg cells, such products would be found in the feces of specific pathogen-free (SPF) mice with a normal spectrum of commensal microorganisms, but not microbiota-deficient mice treated with broad-spectrum antibiotics (AVNM) or germ-free (GF) mice. Polar solvent extracts of feces from SPF, but not GF or AVNM-treated mice potentiated induction of Foxp3 upon stimulation of purified peripheral naïve (CD44loCD62LhiCD25−) CD4+ T cells by CD3 antibody in the presence of dendritic cells (DCs), IL-2, and TGF-β—as depicted in FIG. 1a).

Short-chain fatty acids (SCFA) were found among bacterial metabolites, and their content was evaluated in fecal extracts from SPF, GF or AVNM-treated mice for their ability to affect Treg cell generation. Analysis of hydrazine-derivatized SCFA by HPLC showed a sharp reduction in propionate and butyrate in extracts from GF and AVNM-treated vs. SPF animals (FIG. 1b). Concentrations of these SCFA in extracts were in a 5 mM range, corresponding to ˜100-125 μM in in vitro Foxp3 induction assays.

Purified butyrate, isovalerate and propionate, but not acetate, augmented TGF-β-dependent generation of Foxp3+ cells in vitro (FIG. 1c).

To exclude the possibility that butyrate allowed for expansion of a few contaminating Treg cells in the starting naïve CD4+ T cell population, mice lacking an intronic Foxp3 enhancer CNS1 were studied. These mice are selectively impaired in extrathymic Treg cell differentiation while thymic differentiation is intact (Josefowicz, S. Z. et al. Nature, 2012, 482, 395-U1510; Zheng, Y. et al. Nature, 2010, 463, 808-12). Butyrate failed to rescue the impaired Foxp3 induction in naïve CD4+ T cells in the absence of CNS1 (FIG. 1d). Consistent with this result, butyrate did not diminish either qualitatively or quantitatively the TGF-β dependence of Foxp3 induction in CNS1-sufficient CD4+ T cells. These data suggested that butyrate promotes extrathymic differentiation of Treg cells.

### Example 3

### Butyrate Provision Promotes Extrathymic Treg Cell Generation In Vivo

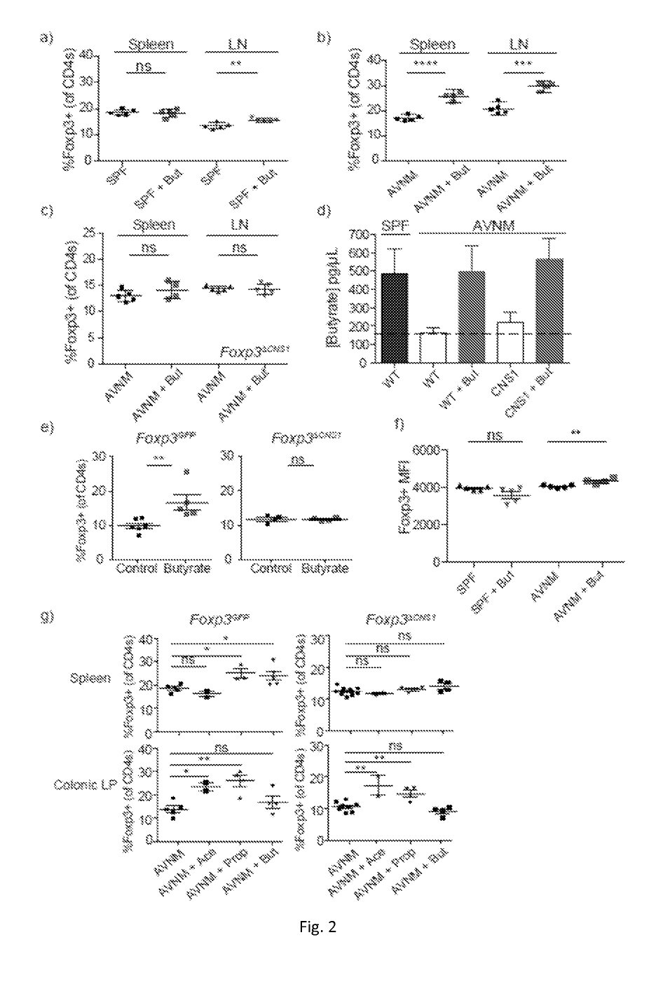

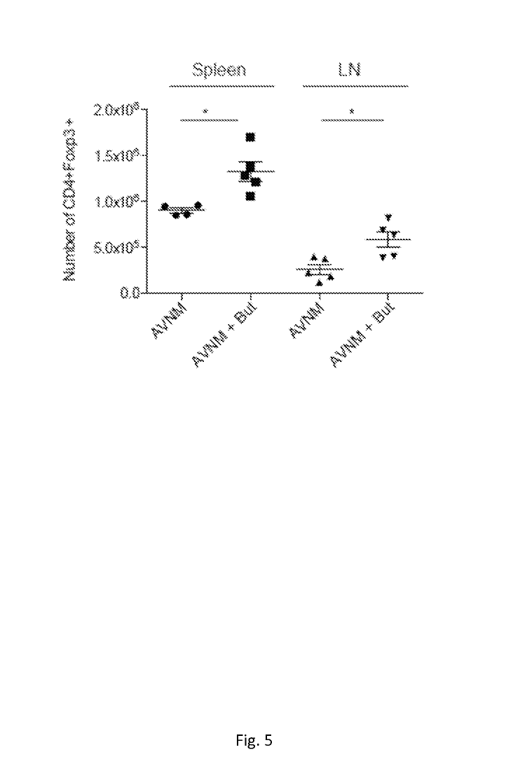

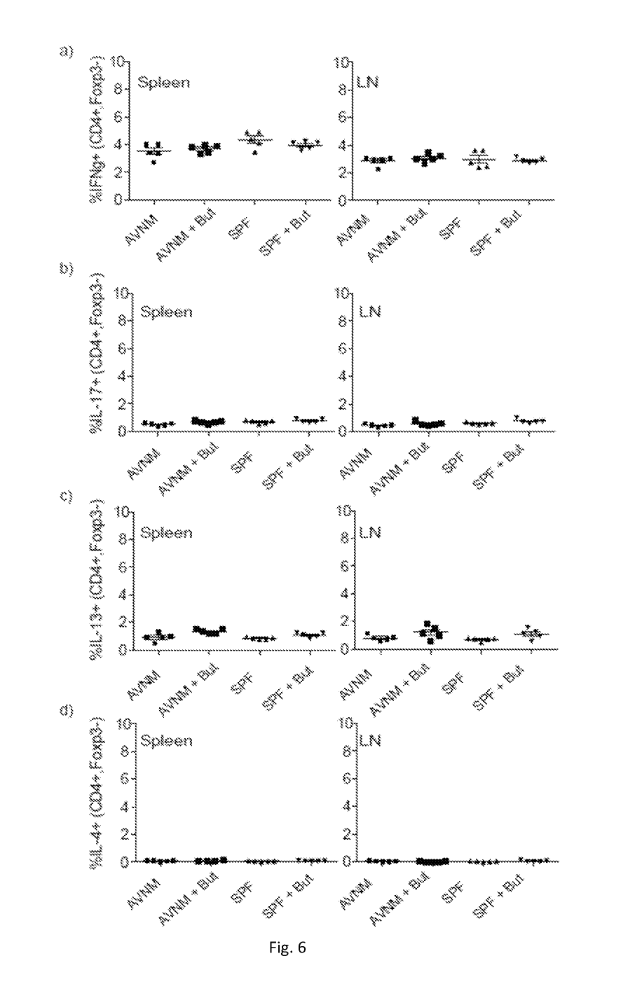

In order to determine if butyrate is capable of promoting extrathymic Treg cell generation in vivo, butyrate was administered in drinking water to AVNM-treated mice, which exhibit a sharp decrease in microbially-derived SCFAs, or untreated control SPF mice. Provision of butyrate to AVNM-treated animals resulted in a robust increase in peripheral, but not thymic or colonic Treg cells (FIG. 2a, 2b, FIG. 5). This increase was not an indirect consequence of an inflammatory response because non-lymphoid tissue histology and production of Th1, Th2, and Th17 cytokines by Foxp3− CD4+ T cells remained unchanged upon butyrate treatment (FIG. 6).

In agreement with the observed CNS1 dependence of in vitro Foxp3 induction, provision of butyrate to AVNM-treated CNS1-deficient mice did not increase the proportion or absolute numbers of Treg cells (FIG. 2c). Thus, the observed butyrate-mediated increase in the Treg cell subset in vivo was due to increased extrathymic generation of Treg cells and not due to their increased thymic output (Josefowicz, S. Z. et al. Nature, 2012, 482, 395-U1510; Zheng, Y. et al. Nature, 2010, 463, 808-12).

To ensure that butyrate reconstitution did not result in its non-physiologically high levels, LC-MS was used to compare amounts of butyrate in the serum of AVNM-treated mice that received butyrate versus amounts found in control SPF mice. While virtually undetectable in AVNM-treated CNS1-sufficient and -deficient animals, butyrate provision resulted in serum levels comparable to those found in unperturbed SPF mice that did not receive butyrate (FIG. 2d). Consistent with the aforementioned unchanged colonic Treg cell subset in AVNM-treated mice that received butyrate via drinking water, levels of butyrate in fecal pellets were not reconstituted in these mice, likely due to its uptake in the small intestine or stomach.

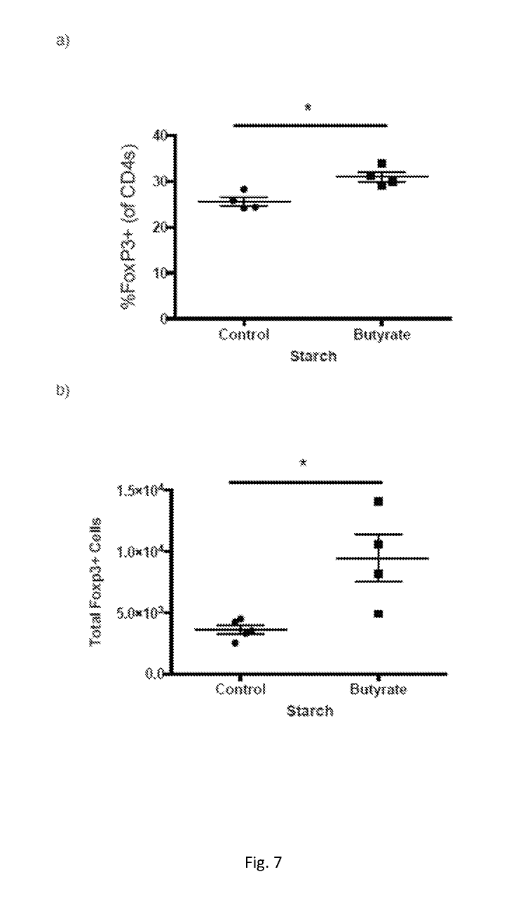

In contrast, delivery of butyrate via enema into the colon of CNS1-sufficient, but not CNS1-deficient mice, led to an increase in the Treg cell subset in the colonic lamina propria (LP) (FIG. 2e). Thus, local provision of butyrate promoted CNS1-dependent extrathymic generation of Treg cells in the colon. Furthermore, feeding mice butyrylated starch, in the absence of antibiotic treatment, increased colonic Treg cell subsets in comparison to a control starch diet (FIG. 7) (Annison, G. et al. J Nutr, 2003, 133, 3523-8).

In addition to increasing Treg cell numbers, restoration of butyrate levels in AVNM-treated animals did not decrease, but instead increased intracellular Foxp3 protein amounts on a per cell basis in both CNS1-sufficient and -deficient mice, suggesting that this bacterial metabolite might also buttress pre-existing Treg cell populations via stabilization of Foxp3 protein expression (FIG. 2f).

In contrast to butyrate's ability to increase Treg cell generation in the colon only upon local, but not systemic delivery, other SCFA, namely acetate and propionate, were recently shown to promote accumulation of Treg cells in the colon by activating GPR43 (Smith, P. M. et al. Science, 2013, 341, 569-73). These results suggested discrete modes of action of these three SCFAs. To test this idea, propionate and acetate were administered in the drinking water to AVNM-treated CNS1-sufficient and -deficient mice. Similarly to butyrate, oral provision of propionate increased Treg cell subsets in the spleen in AVNM-treated CNS1-sufficient, but not -deficient animals, suggesting that propionate also promotes de novo generation of peripheral Treg cells (FIG. 2g). In contrast, acetate did not increase splenic Treg cell numbers. These results were fully consistent with in vitro Treg cell differentiation studies (FIG. 1c). In the colon, however, both acetate and propionate, but not butyrate promoted accumulation of Treg cells in a CNS1-independent manner (FIG. 2g). These results suggest that butyrate promotes de novo generation, but not colonic accumulation of Treg cells, whereas acetate has a diametrically opposite activity and propionate is capable of both.

### Example 4

### Butyrate Acts within T Cells to Enhance Acetylation of the Foxp3 Locus and Foxp3 Protein

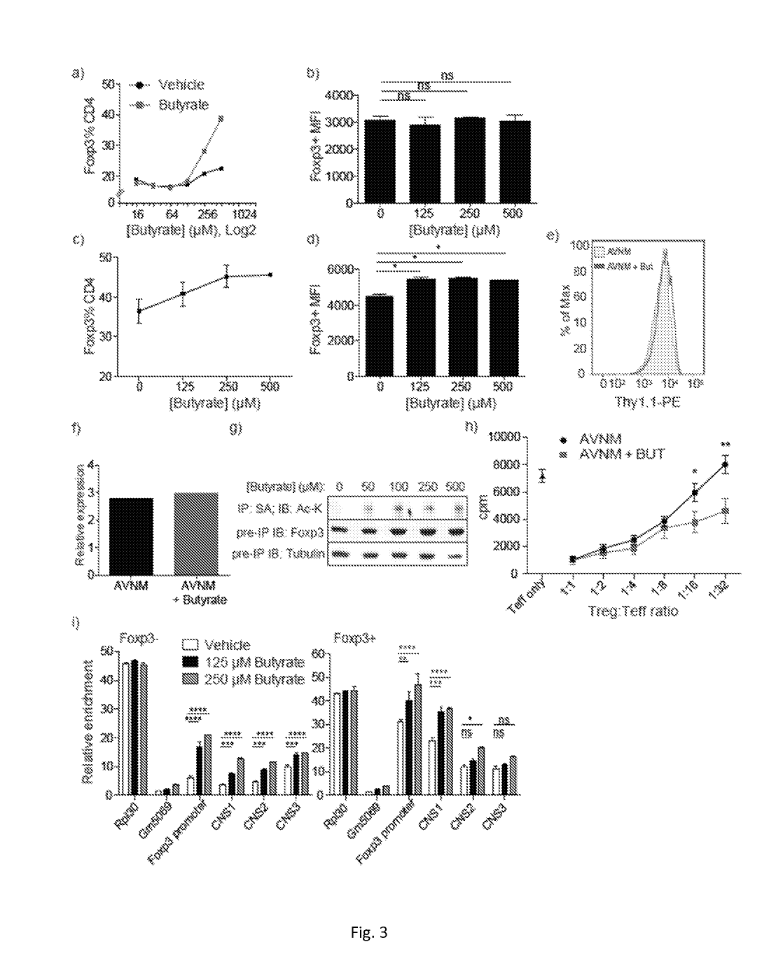

The observation that butyrate facilitates extrathymic differentiation of Treg cells raised a question as to whether butyrate directly affects T cells or DCs (or both) by enhancing their ability to induce Foxp3 expression. To explore these non-mutually exclusive possibilities, the effects of butyrate on the ability of T cells and DCs to generate Treg cells in vitro were assessed (FIG. 3a-d). Butyrate increased, (<1.5-fold), the numbers of Foxp3+ cells in DC-free cultures of purified naive CD4+ T cells stimulated by CD3 and CD28 antibody-coated beads and TGF-β (FIG. 3c). Like Treg cells isolated from butyrate-treated mice, Treg cells generated in the presence of butyrate in vitro expressed not lower, but rather higher amounts of Foxp3 protein on a per-cell basis than those from butyrate-free cultures (FIG. 3d). This effect was not associated with increased Foxp3 mRNA levels (FIG. 3e, f). Instead, it was likely due to increased Foxp3 protein acetylation observed in the presence of butyrate, a known histone deacetylase (HDAC) inhibitor (FIG. 3g). Foxp3 acetylation confers greater stability and enhanced function (van Loosdregt, J. et al. PLoS One, 2011, 6, e19047; van Loosdregt, J. et al. Blood, 2010, 115, 965-74; Zhang, H., et al. Immunol Cell Biol, 2012, 90, 95-100; Song, X. et al. Cell Rep, 2012, 1, 665-75). Furthermore, the suppressor activity of Treg cells isolated from mice treated with AVNM and butyrate was not attenuated, but was moderately enhanced as compared to mice treated with AVNM alone (FIG. 3h).

Previous in vitro studies suggested that a synthetic HDAC inhibitor, trichostatin A (TSA), potentiates Treg cell generation in vitro by acting on T cells (Wang, L., et al. Nat Rev Drug Discov, 2009, 8, 969-81; Tao, R. et al. Nat Med, 2007, 13, 1299-307). Since butyrate can also boost extrathymic Treg cell generation by acting directly on T cells in the absence of DCs (FIG. 3c), the effect of butyrate on histone modification at the Foxp3 locus was assessed. When naïve CD4+ T cells from Foxp3GFP mice were stimulated by CD3 and CD28 antibody-coated beads and TGF-β with or without butyrate for 3 days, a marked 3-fold increase in H3K27-Ac at the Foxp3 promoter and CNS1 enhancer was observed in Foxp3− cells purified from these cultures (FIG. 3i). In contrast, increases in the H3K27-Ac occupancy in their Foxp3+ counterparts was expectedly minor (˜30%) and inconsequential. Accordingly, Foxp3 mRNA levels were not different in Foxp3+ cells in the presence or absence of butyrate (FIG. 3e,f). Although difficult to discriminate between the contribution of increased acetylation of histone vs. non-histone targets to heightened Foxp3 induction, it is likely facilitated by the increase in H3K27-Ac observed in Foxp3− T cells.

### Example 5

### HDAC-Inhibitory Activity of Butyrate Decreases Pro-Inflammatory Cytokine Expression within DC to Promote Treg Induction

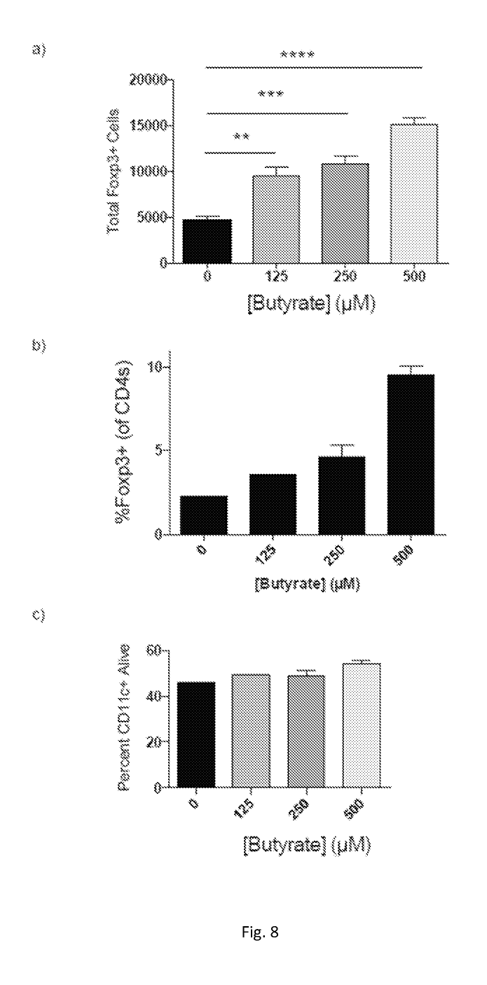

In addition to its direct Treg cell differentiation-promoting effects on CD4+ T cell precursors, butyrate endowed DCs with a superior ability to facilitate Treg cell differentiation. Pretreatment of DCs with butyrate in vitro for 6 h followed by its removal markedly enhanced their ability to induce Foxp3 expression in naïve CD4+ T cells stimulated by CD3 antibody and TGF-β in the absence of butyrate (FIG. 3a; FIG. 8a, b). The latter treatment had no detrimental effect on DC viability (FIG. 8c). The Foxp3 protein expression induced by butyrate-pretreated and control DCs was comparable, in contrast to a T cell-intrinsic effect of butyrate leading to increased amounts of Foxp3 protein in Treg cells in mice treated with AVNM and butyrate (FIG. 3b, FIG. 2f).

### Example 6

### Butyrate Increases Numbers of Treg Cells In Vivo

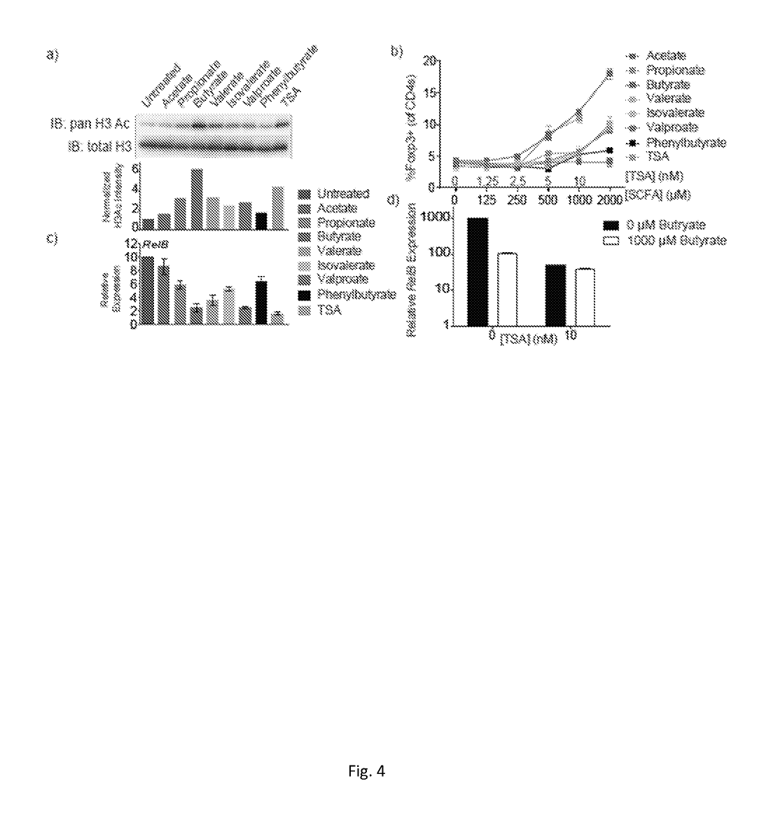

The HDAC inhibitory activity of butyrate and other SCFAs in DCs using histone H3 acetylation as an indirect readout was tested (FIG. 4a). TSA and valproate, two chemically distinct HDAC inhibitors, and phenylbutyrate, a butyrate analog with a relatively weak inhibitory activity, were used as controls in these experiments. Relative HDAC inhibitory activity of SCFAs closely correlated with their ability to potentiate the capacity of DCs to induce Treg cell differentiation. DCs briefly exposed to butyrate, TSA, and to a lesser extent propionate, but not acetate, potently induced Foxp3 expression (FIG. 4b).

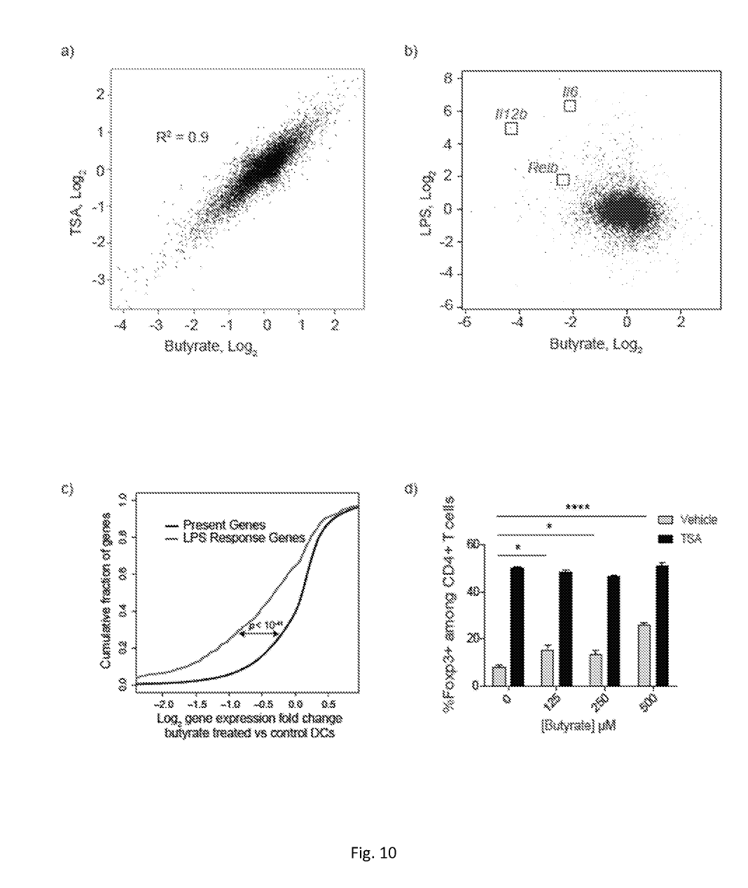

Furthermore, microarray analysis showed that butyrate and TSA induced remarkably similar, if not identical, gene expression changes in DCs (FIG. 10a) with a systemic repression of LPS response genes including 1112, 116, and Relb (FIG. 10b,c).

Repression of Relb, a major inducer of DC activation, correlated with the level of HDAC-inhibitory activity of butyrate and other SCFAs (FIG. 4c) (MacDonald, K. P. et al. Blood, 2007, 109, 5049-57). Notably, knockdown of RelB in DC promotes their ability to support Treg cell differentiation (Zhu, H. C. et al. Cell Immunol, 2012, 274, 12-8; Shih, V. F. et al. Nat Immuno 2012, 13, 1162-70). To further ascertain that TSA and butyrate potentiated Treg cell generation through HDAC inhibition and not through distinct independent mechanisms, DCs were treated with the combination of butyrate and optimal amounts of TSA. If butyrate and TSA were to act via independent mechanisms, they should have exhibited synergistic effects on Foxp3 induction. However, if they acted on identical or related targets, i.e. HDACs, additive effects were unlikely. In support of the latter scenario, butyrate was unable to further enhance the ability of TSA to down-regulate Relb and promote Foxp3 induction (FIG. 4d; FIG. 10d). These results are consistent with the idea that the HDAC inhibitory activity of butyrate as well as propionate contributes to the ability of DCs to facilitate extrathymic Treg cell differentiation.

### Example 7

### GPCR Sensing and Butyrate Transporters are not Required for Butyrate-Dependent Increase in Treg Cell Induction by Dendritic Cells

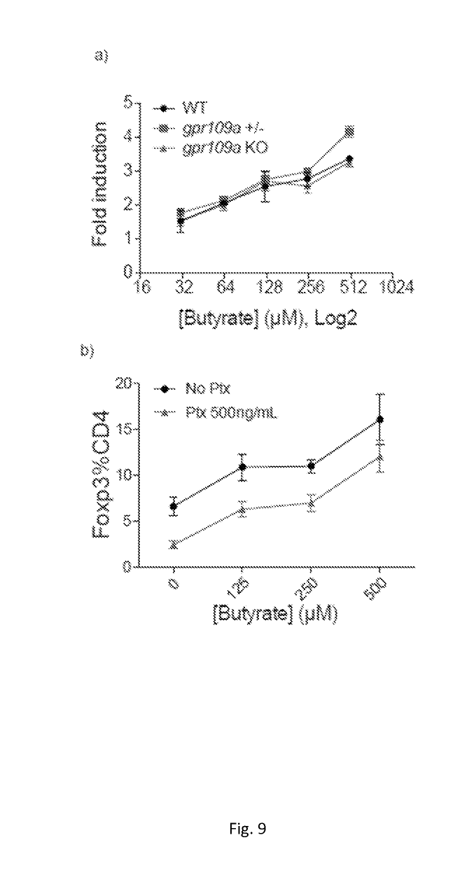

Butyrate sensing by G protein-coupled receptors (GPR) was considered as a potential mechanism for the increase in extrathymic differentiation of Treg cells (Maslowski, K. M. et al. Nature, 2009, 461, 1282-U119; Thangaraju, M. et al. Cancer Res 2009, 69, 2826-32). However, pre-treatment of Gpr109a+/+ and Gpr109a−/− DCs with butyrate similarly increased in vitro generation of Foxp3+ cells (FIG. 9a). Consistent with these results, butyrate-dependent potentiation of Foxp3 induction by DCs remained unchanged upon pretreatment with pertussis toxin (FIG. 9b) (Nilsson, N. E., et al. Biochem Biophys Res Commun, 2003, 303, 1047-52).

### Example 8

### Liposome Mediated Delivery of HDAC Inhibitors to Dendritic Cells Promotes Treg Induction

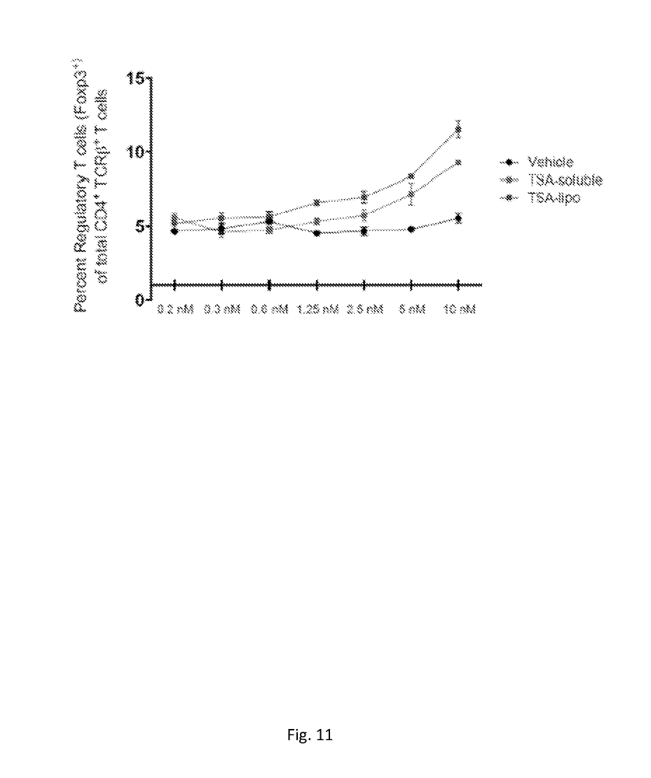

The ability of dendritic cells to induce Treg development in vitro after exposure to lipopsome encapsulated TSA was evaluated. Doubly bead-purified dendritic cells were treated for 6 hrs with soluble or liposome encapsulated TSA then washed and incubated with naïve CD4+ T cells. Pretreatment of DCs with TSA increased the generation of Foxp3+ cells relative to vehicle control (FIG. 11). These data further confirm, as demonstrated herein, that dendritic cells exposed to HDAC inhibitors are endowed with the ability to induce Foxp3 expression in naïve CD4+ T cells.

### Example 9

### Provision of Butyrate Through Starch Diet Increases Butyrate but not Propionate in Colon

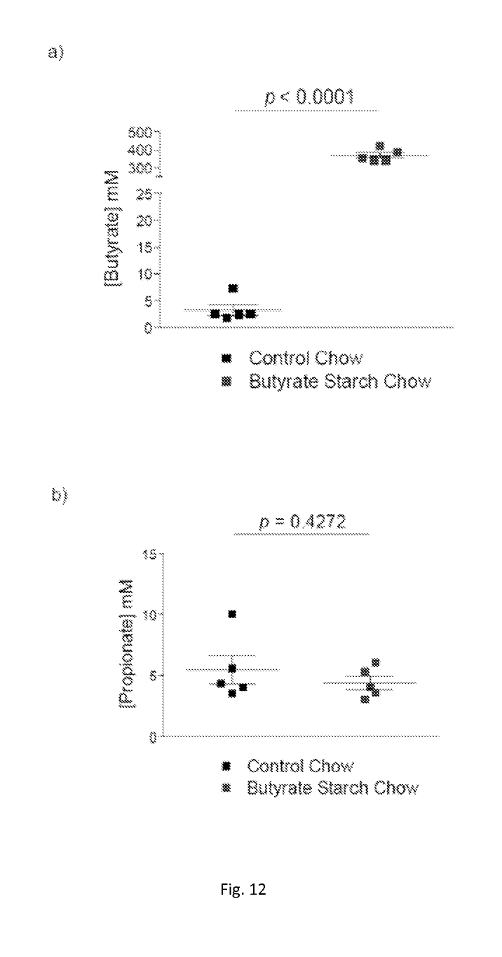

Local increase of SCFA in the colon was measured. Mice were fed control chow or chow containing 15% butyrate-modified starch. Fecal pellets were collected after 3 weeks and quantified for the presence of SCFA by ¹H NMR. The butyrate starch diet increased butyrate levels in the colon but not propionate (FIG. 12) These data further confirm, as demonstrated herein, that a starch diet can increase specific SCFA concentration in the colon.

### OBSERVATIONS

These Examples suggest that butyrate and propionate, produced by commensal microorganisms, increased extrathymic CNS1-dependent differentiation of Treg cells. The Examples indicate that metabolic by-products of commensal microorganisms influence the balance between pro- and anti-inflammatory cells and serve as a means of communication between the commensal microbial community and the immune system.

Without wishing to be held to a particular theory, the above Examples suggest that targeting Treg cells is likely to result in pronounced clinical responses to diseases or disorders associated with inflammation.

What is claimed is:

1. A method of inducing differentiation of regulatory T cells (Treg), the method comprising steps of:
administering to cells undergoing inflammation a composition comprising a histone deacetylase (HDAC) inhibitory agent.

2. The method of claim 1, wherein the method comprises the step of:
administering to dendritic cells and naïve CD4⁺ T cells a composition comprising a histone deacetylase (HDAC) inhibitory agent.

3. The method of claim 1, wherein the step of:
administering comprises administering a composition comprising a histone deacetylase (HDAC) inhibitory agent that acts to promote Treg differentiation in a Foxp3-dependent manner.

4. The method of claim 1, wherein the step of:
administering comprises administering a composition selected from the group comprising bacterial metabolites, short chain fatty acids, histone deacetylase (HDAC) inhibitors, and analogs thereof.

5. The method of claim 1, wherein the step of:
administering comprises administering a composition comprising short chain fatty acids.

6. The method of claim 1, wherein the step of:
administering comprises administering short chain fatty acids selected from the group comprising butyrate, propionate, succinate, formate, valproate, phenylbutyrate, L-lactate, 2-ethylbutyrate, isovalerate, isobutyric acid, valeric acid, and acetate and analogs thereof.

7. The method of claim 1, wherein the step of administering comprises administering to a site or individual suffering from or susceptible to inflammation and furthermore comprises administering the composition by a route according to a schedule so that inflammation is reduced.

8. The method of claim 1, wherein the step of administering comprises administering to a subject suffering from or susceptible to a Treg-associated Disease, Disorder or Condition.

9. The method of claim 8, wherein the Treg-associated Disease, Disorder or Condition is selected from the group comprising colitis, asthma, chronic peptic ulcer, tuberculosis, rheumatoid arthritis, chronic periodontitis, Crohn's disease, chronic sinusitis, pelvic inflammatory disease, hepatitis, inflammatory bowel disease, sarcoidosis, vasculitis, celiac disease, autoimmune disease, reperfusion injury, transplant rejection, diabetes and infection.

10. The method of claim 1, wherein the step of administering comprises administering by a route selected from intradermal, intramuscular, intraoperative, intrathecal, intravenous, nasal, ocular, oral, parental, rectal, subcutaneous, topical, and transdermal.

11. A method comprising steps of:
administering to a subject in need thereof a pharmaceutical composition comprising an activating agent that is or increases the level of a short chain fatty acid to induce Treg differentiation in a subject.

12. A method comprising the steps of:
administering to a subject in need thereof a pharmaceutical composition comprising an activating agent that is or increases the level of a bacterial metabolite selected from the group consisting of short chain fatty acid.

13. The method of claim 12, wherein the pharmaceutical composition delivers an amount of the active agent that is effective, when the composition is administered in accordance with a therapeutic regimen, to increase level or activity of pTreg cells.

14. The method of claim 13, wherein the increase level or activity of pTreg cells occurs at local sites of pTreg cells.

15. The method of claim 13, wherein the increase level or activity of pTreg cells occurs in pTregs systemically throughout the organism.

16. The method of claim 1, wherein the step of:
administering comprises administering compositions to treat an animal.

17. The method of claim 16, wherein the step of:
administering comprises administering compositions to treat a mammal.

18. A method of identifying or characterizing a pTreg stimulatory agent, the method comprising steps of:
administering the candidate pTreg stimulatory agent to a T cell population;
comparing the pTreg level and/or activity to a reference set of positive control conditions and negative control conditions; and
determining whether the pTreg level and/or activity is at least comparable to positive control conditions and/or higher than that under (other) negative control conditions.

19. An agent characterized in that when administered to T cells will induce differentiation of Treg cells.

20. The agent of claim 19, wherein the agent acts to promote Treg differentiation in a Foxp3-dependent manner

21. The agent of claim 19, wherein the agent is selected from a group comprising bacterial metabolites, short chain fatty acids, histone deacetylase (HDAC) inhibitors and analogs thereof.

22. The agent of claim 21, wherein the short chain fatty acid is selected from the group comprising butyrate, propionate, succinate, formate, valproate, phenylbutyrate, L-lactate, 2-ethylbutyrate, isovalerate, isobutyric acid, valeric acid, and acetate and pharmaceutically acceptable salts and analogs thereof.
